(19) United States
(12) Patent Application Publication
(10) Pub. No.: US 2025/0034538 A1
(43) Pub. Date: Jan. 30, 2025

(54) FUSION PROTEINS TARGETED TO THE CENTRAL NERVOUS SYSTEM

(71) Applicant: Hoffmann-La Roche Inc., Little Falls, NJ (US)

(72) Inventors: Per-Ola Freskgard, Norrköping (SE); Stefan Frost, Bad Heilbrunn (DE); Alexandra Maria Gehrlein, Sierentz (FR); Ravi Jagasia, Basel (CH); Jens Niewoehner, Neuried (DE); Ralf Thoma, Grenzach-Wyhlen (DE)

(73) Assignee: Hoffmann-La Roche Inc., Little Falls, NJ (US)

(21) Appl. No.: 18/911,752

(22) Filed: Oct. 10, 2024

Related U.S. Application Data
(63) Continuation of application No. PCT/EP2023/059358, filed on Apr. 11, 2023.

(30) Foreign Application Priority Data
Apr. 12, 2022 (EP) 22167801.4
Jun. 3, 2022 (EP) 22177133.0

(51) Int. Cl. C12N 9/24; A61K 38/00; A61P 25/28

(52) U.S. Cl. CPC C12N 9/2402; A61P 25/28; A61K 38/00; C07K 2319/30; C12Y 302/01045

(57) ABSTRACT

The present invention relates to fusion proteins targeted to the central nervous system (CNS) and its use for the treatment of lysosomal storage disorders (LSD).

This application is a continuation of International Application No. PCT/EP2023/059358, filed Apr. 11, 2023, which claims benefit of priority to European Patent Application No. 22167801.4 filed Apr. 12, 2022 and European Patent Application No. 22177133.0 filed Jun. 3, 2022, each of which is incorporated herein by reference in its entirety.

This application contains a Sequence Listing which has been submitted electronically in XML format and is hereby incorporated by reference in its entirety. Said XML copy, cre-ated on Oct. 7, 2024, is named P37329-US_SL.xml and is 42,426 bytes in size.

The present invention relates to fusion proteins targeted to the central nervous system (CNS) and its use for the treatment of lysosomal storage disorders (LSD).

Lysosomes host more than 60 soluble lysosomal hydrolases and accessory proteins, as well as over 120 lysosomal membrane proteins and transitory protein residents¹. Dysfunction in some of these proteins lead to lysosomal storage disorders (LSDs), which collectively have a relatively high incidence in the general population: more than 1:5000 live births are affected by a LSD². Notable among these lysosomal enzymes is β-glucocerebrosidase (GCase), which is encoded by GBA, and is responsible for the hydrolytic release of glucose from the glycolipids glucosylsphingosine (GlcSph) and glucosylceramide (GlcCer). Deficiency in GCase enzymatic activity leads to an accumulation of these glycolipids causing lysosomal storage disorders with varying disease severity³. Neuronopathic Gaucher's disease (GD), type 2 results from severe or null mutations in GBA1 and is ultimately lethal. Currently, there is no medica-tion that alters its disease trajectory⁴⁻⁷. While often debilitating, chronic neuronopathic or type 3 GD has varying degrees of neurological manifestations, but patients survive infancy and can present late⁸. Both homozygous and heterozygous carriers of mutant GBA1 alleles are at increased risk for sporadic and complex neurodegenerative diseases including Parkinson's disease (PD) and dementia with Lewy Bodies (DLB). Here too, there is no disease-mod-ifying therapy that slows the trajectory of the disease. GBA1-associated PD (GBA-PD), while neuropathologically indistinguishable from sporadic PD, is often associated with an earlier disease onset, more pronounced non-motor symptoms and a faster disease progression^{9,10}. It is hoped that treatment paradigms targeting impaired GCase to restore its intracellular lysosomal function will prove beneficial for disorders ranging from neuronopathic GD to neurodegenerative diseases such as PD and DLB.

GD- and PD-associated pathologic variants in GBA1, for example, L444P (p.L483P) and N370S (p.N409P) lead to the production of misfolded mutant enzymes with significantly reduced activity, in the range of 10 to 20% of normal 11. The accumulation of glycosphingolipids, which result from lowered GCase activity, is a key pathological event in GD and may be a triggering event for the neurodegeneration associated with PD 12-14. Thus, it is very likely, given the shared genetics, that increasing GCase lysosomal activity in the brain would be a viable therapy to restore lysosomal homeostasis in both neuronopathic GD and GBA1-associated neurodegenerative diseases.

Currently available treatments for GD include substrate reduction therapy using small molecules or enzyme replacement therapy (ERT). Both, however, fail to target GCase deficiency within the CNS. Therefore, there is a need for molecules and therapies targeting the CNS aspects of LSDs.

In a first aspect, the present invention provides a fusion protein comprising:
a lysosomal protein, a Fc region of an antibody and an antibody fragment targeting the transferrin receptor, wherein the antibody fragment has a monovalent binding mode.

In an embodiment of the present invention, the lysosomal protein is a β-Glucocerebrosidase (Gcase) protein, preferably a human Gcase protein or a variant thereof.

In an embodiment of the present invention, the Fc region of an antibody is the Fc region of an IgG antibody, preferably an IgG1 antibody.

In an embodiment of the present invention, the Fc region is devoid of Fc receptor gamma binding.

In an embodiment of the present invention, the antibody fragment targeting the transferrin receptor is selected from the group consisting of Fv, Fab, Fab′, Fab′-SH, F(ab′) diabodies, linear antibodies, single-chain antibody molecule such as e.g. scFv, scFab, cross Fab and single domain antibodies (dAbs).

In an embodiment of the present invention, one chain of the Fc region is fused at its N-terminal end to the C-terminal end of the lysosomal protein and the second Fc chain is fused at its C-terminal end to the antibody fragment targeting the transferrin receptor.

In an embodiment of the present invention, the two Fc chains form a dimer using the knob-into-hole technology.

In an embodiment of the present invention, the fusion protein comprises two protein chains:
1. the first protein chain comprising the lysosomal protein fused at its C-terminal end to a first chain of the Fc region comprising the knob-into-hole technology,
2. the second protein chain comprising the second chain of the Fc region comprising the knob-into-hole technology fused at its C-terminal end to the scFab antibody fragment targeting the transferrin receptor.

In an embodiment of the present invention, the human Gcase protein has the amino acid sequence set forth in Seq. Id. No. 1.

In an embodiment of the present invention, the first protein chain has the amino acid sequence set forth in Seq. Id. No. 2 and the second single chain protein has the amino acid sequence set forth in Seq. Id. No. 3.

In a second aspect, the present invention relates to an isolated nucleic acid molecule encoding the fusion protein of the present invention.

In an embodiment of the present invention, the nucleic acid is a circular RNA.

In a third, aspect the present invention relates to a host cell comprising the isolated nucleic acid molecule of the present invention.

In a fourth aspect, the present invention provides a pharmaceutical formulation comprising the fusion protein of the present invention.

In an additional aspect, the present invention provides the fusion protein of the present invention as a medicament.

In an additional aspect, the present invention relates to the use of the fusion protein of the present invention for the treatment of a neurodegenerative disorder, in particular a LSD, more particular the CNS aspects of a LSD.

In an additional aspect, the present invention relates to a recombinant AAV vector comprising the nucleic acid molecule encoding the fusion protein of the present invention.

In an embodiment, the present invention relates to an AAV virus particle comprising the AAV vector of the present invention.

In an embodiment of the present invention relates to a pharmaceutical composition comprising an AAV virus particle of the present invention.

In an additional embodiment, the present invention provides the use of the AAV virus particle of the present invention for the treatment of a neurodegenerative disorder, in particular LSD, more particular the CNS aspects of a LSD.

### Definitions

The term “lysosomal protein” refers to proteins which are localized in Lysosomes including more than 60 soluble lysosomal hydrolases and accessory proteins, as well as over 120 lysosomal membrane proteins and transitory protein residents.

Herein, “GCase” is an abbreviation used for β-glucocerebrosidase. Human β-Glucocerebrosidase has the Uniprot ID: P04062. The amino acid sequence of mature, human GCase variant R534H is set forth in Seq. Id. No. 1. Mature, human GCase comprises amino acids 40-536 of the human GCase with Uniprot ID: P04062. Furthermore, variant, recombinant β-glucocerebrosidase proteins herein also include functional fragments or derivatives thereof.

The “blood-brain barrier” or “BBB” refers to the physiological barrier between the peripheral circulation and the brain and spinal cord (i.e., the CNS) which is formed by tight junctions within the brain capillary endothelial plasma membranes, creating a tight barrier that restricts the transport of molecules into the brain, even very small molecules such as urea (60 Daltons). The blood-brain barrier within the brain, the blood-spinal cord barrier within the spinal cord, and the blood-retinal barrier within the retina are contiguous capillary barriers within the CNS, and are herein collectively referred to a the blood-brain barrier or BBB. The BBB also encompasses the blood-CSF barrier (choroid plexus) where the barrier is comprised of ependymal cells rather than capillary endothelial cells.

The “transferrin receptor” (“TfR”) is a transmembrane glycoprotein (with a molecular weight of about 180,000) composed of two disulphide-bonded sub-units (each of apparent molecular weight of about 90,000) involved in iron uptake in vertebrates. In one embodiment, the TfR herein is human TfR comprising the amino acid sequence as in Schneider et al. Nature 311:675-678 (1984), for example.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyri-bonucleic acid (DNA) including e.g., complementary DNA (cDNA) and genomic DNA, ribo-nucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, pub-lished online 12 Jun. 2017, doi: 10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Percent (%) amino acid sequence identity.” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for in-stance, using publicly available computer software such as BLAST, BLAST-2, Clustal W. Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448: W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta. bioch.vir-ginia.edu/fasta_www2/index.cgi can be used to compare the sequences, using the ggsearch (global protein: protein) program and default options (BLOSUM50); open: −10; ext: −2: Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are un-acceptably toxic to a subject to which the pharmaceutical composition would be adminis-tered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabi-lizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay develop-ment of a disease or to slow the progression of a disease.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal gly-cine (Gly 446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

An “antibody fragment” refers to a molecule other than an intact antibody that comprises a portion of an intact antibody that binds the antigen to which the intact antibody binds. Examples of antibody fragments include but are not limited to Fv. Fab, Fab′, Fab′-SH, F(ab′)2; diabodies; linear antibodies: single-chain antibody molecules (e.g., scFv, and scFab): single domain antibodies (dAbs); and multispecific antibodies formed from antibody fragments. For a review of certain antibody fragments, see Holliger and Hudson, Nature Biotechnology 23:1126-1136 (2005). In a particular embodiment antibody fragments are anti-transferrin receptor antibodies such as e.g. disclosed in WO 2014/033074 and WO 2012/075037. In a particular embodiment, the antibody fragment is a Fab antibody fragment or a scFab antibody fragment directed to the human transferrin receptor, preferably a cross Fab antibody fragment. Exemplary cross Fab fragments are described in WO 2009/080251, WO 2009/080252 and MABS 2016, VOL. 8. NO. 6, 1010-1020.

The “monovalent binding mode” refers to a specific binding to the TfR where the interaction between the antibody fragment and the TfR take place through one single epitope. The monovalent binding mode prevents any dimerization/multimerization of the TfR due to a single epitope interaction point. The monovalent binding mode prevents that the intracellular sorting of the TfR is changed.

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)).

An “AAV vector” as used herein refers to a vector comprising one or more polynucleo-tides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV viral particle” refers to a viral particle composed of at least one AAV capsid protein and an encapsidated polynucleotide AAV vector. If the particle comprises a heterolo-gous polynucleotide (i.e. a polynucleotide such as a transgene to be delivered to a mammalian cell), it is typically referred to as an “AAV vector particle” or simply an “AAV vector”.

### DESCRIPTION OF THE DRAWINGS

The patent or application file contains at least one drawing executed in color. Copies of this patent or patent application publication with color drawing(s) will be provided by the Office upon re-quest and payment of the necessary fee.

FIGS. 1A-1E: Purified GCase-BS molecules are fully functional.

FIG. 1A: Schematic of GCase-BS depicting different moieties.

FIG. 1B: Assessment of mouse transferrin receptor (mTfR) binding of mGCase-mBS by FACS analysis of mTfR-expressing cells.

FIG. 1C: Assessment of human transferrin receptor (hTfR) binding of hGCase-hBS by FACS analysis of hTfR-expressing cells. Specificity of TfR-binding moiety was shown for the mBS as no signal was observed in hTfR-expressing cells.

FIG. 1D: Enzymatic activity of various GCase(-BS) molecules (Michaelis-Menten-dia-gram). Resorufin-β-glucopyranoside was used as substrate.

FIG. 1E: Enzymatic activity and IgG levels measured for both mGCase-mBS and hGCase-hBS after 15 min.

FIGS. 2A-2F: Brain shuttle module improves cellular uptake and lysosomal efficacy in vitro.

FIG. 2A: Total GCase activity in mouse cortical neurons as a measure of cellular uptake after 2 h of treatment with imiglucerase, mGCase or mGCase-mBS. Data was normalised to Gba+/+ cells.

FIG. 2B: Total GCase activity in H4 cells as a measure of cellular uptake after 2 h of treatment with Cer, hGCase-hBS-NB or hGCase-hBS. Data was normalised to GBA+/+ cells.

FIG. 2C: Live imaging of GCase activity (FQ-7) and lysosomes (SiR lyso) and quantification of co-localising signal in mouse cortical neurons. Data was normalised to GBA+/+ cells.

FIG. 2D: Live imaging of GCase activity (FQ-7) and lysosomes (SiR lyso) and quantification of co-localising signal in H4 cells. Data was normalised to GBA+/+ cells.

FIG. 2E: Glucosylsphingosine measurement in mouse cortical neurons as a measure of lysosomal efficacy after 48 h of treatment with Cer, mGCase or mGCase-mBS. Data was normalised to GBA−/−cells.

FIG. 2F: Glucosylsphingosine measurement in H4 cells as a measure of lysosomal efficacy after 48 h of treatment with Cer, hGCase-NB or hGCase-hBS. Data was normalised to GBA−/−cells.

Bar graphs represent group means+SEM. n=3. Activity data were analysed by two-way ANOVA (Tukey's multiple comparisons test). ** p<0.01: *** p<0.001: **** p<0.0001.

FIGS. 3A-3H: GCase-BS lysosomal mode of action in vitro.

FIG. 3A: Immunolabeling of hBS and colocalisation with LAMPI upon acute treatment with various BS constructs +/−fusion to hGCase. Colocalising hBS spots were quantified and normalised to total amount of LAMPI spots. n=5.

FIG. 3B: Immunolabeling of hGCase and colocalisation with LAMPI upon acute treatment with various BS constructs +/−fusion to hGCase. Colocalising GCase spots were quantified and normalised to total amount LAMPI spots.

FIG. 3C: Total GCase activity in GBA-deficient TfR WT and TfR KO neuroblastoma lines.

FIG. 3D: Glucosylsphingosine measurement in GBA-deficient TfR WT and TfR KO neuroblastoma lines.

FIG. 3E: Tofal GCase activity in GBA-deficient M6PR-CI WT and M6PR-CI KO neuroblastoma lines.

FIG. 3F: Glucosylsphingosine measurement in GBA-deficient M6PR-CI WT and M6PR-CI KO neuroblastoma lines.

FIG. 3G: Total GCase activity in GBA-deficient M6PR-CD WT and M6PR-CD KO neuroblastoma lines.

FIG. 3H: Glucosylsphingosine measurement in GBA-deficient M6PR-CD WT and M6PR-CD KO neuroblastoma lines. n=6.

Bar graphs represent mean+SEM. Data were analysed by Student's two-tailed t-test comparing WT and KO of each receptor for each treatment. * p<0.05: ** p<0.01: *** p<0.001, n=6.

FIGS. 4A-4D: hGCase-hBS reverts protein and lipid dysregulation in lysosomes from GBA1 KO H4 cells.

FIG. 4A: Scheme for hGCase-hBS treatment and lysosome isolation from cells. H4 cells expressing a lysosome-tag (TMEM192-3HA) are treated with 1 nM hGCase-hBS for 24 h, followed by cell lysis and lysosome isolation using anti-HA coated magnetic beads.

FIG. 4B: Validation of lysosome enrichment after TMEM192-3HA-based Lyso-IP. Western blots showing enrichment of lysosomes after Lyso-IP as demonstrated by enrichment of bonafide lysosomal proteins Lamp2, Cathepsin D and GCase in eluent fraction compared to input.

FIG. 4C: Rescue of dysregulated proteins upon hGCase-hBS treatment in lysosomes. Graph showing increased or decreased levels of proteins in GBA1 KO lysosomes (blue bar) and its rescue upon hGCase-hBS treatment (green bar).

FIG. 4D: Rescue of dysregulated lipids upon hGCase-hBS treatment in lysosomes. Graph showing increased or decreased levels of lipid species in GBA1 KO lysosomes (blue bar) and its rescue upon hGCase-hBS treatment (green bar).

FIGS. 5A-5B: GCase-BS proof of concept in vivo.

FIG. 5A: PK study in GBA+/+ mice to assess systemic exposure of mGCase-mBS in plasma and brain. n=3 mice per group.

FIG. 5B: Multi-dose PD study in 4L/PS-NA mice to compare equimolar doses of mGCase vs. mGCase-mBS. GlcSph levels were measured in cortex, midbrain and liver. n=6 mice per group.

Data is represented as group mean+/−SEM. Data was analysed by one-way ANOVA (Dunnett's multiple comparisons test) comparing each treatment group to 4L/PS-NA, vehicle. n.s. p>0.05: **** p<0.0001.

FIGS. 6A-6B: GCase-BS longitudinal effects in vivo.

FIG. 6A: Single-dose PD study in 4L/PS-NA mice to inform about duration of GlcSph lowering in cortex and midbrain. GlcSph levels rebounce between 15-30 days post administration. n=4-6 mice per group.

FIG. 6B: Chronic study in 4L/PS-NA mice with monthly or bi-weekly dosing frequency. GlcSph levels in cortex and midbrain as efficiency readout. NfL levels in plasma as readout for neurodegeneration. n=10 mice per group.

Data is represented as group mean+/−SEM. Data was analysed by one-way ANOVA (Dunnett's multiple comparisons test) comparing each treatment group to 4L/PS-NA, vehicle. n.s. p>0.05: * p<0.05: ** p<0.01; *** p<0.001: **** p<0.0001

### DESCRIPTION

### Proteins of the Present Invention

| | Seq. |
|---|---|
| | Id. |
| Protein | No. |
| Human β-Glucocerebrosidase Uniprot ID: P04062; amino acids 40- | 1 |
| 536, variant R534H | |
| Human β-Glucocerebrosidase Uniprot ID: P04062; amino acids 40- | 2 |
| 536, variant R534H fused to human IgG1 Fc region single chain | |
| Human IgG1 Fc single region chain fused to anti-human transferrin | 3 |
| scFab 1026 | |
| Human IgG1 Fc single region chain fused to anti-transferrin DP47 | 4 |
| scFab | |
| Mouse β-Glucocerebrosidase Uniprot ID: P1AE2668, amino acids | 5 |
| 19-515, C-Sortase site-6His | |
| Mouse β-Glucocerebrosidase Uniprot ID: P1AE2668, amino acids | 6 |
| 19-515, fused to human IgG1 Fc region single chain | |
| Human IgG1 Fc single region chain fused fused to anti mouse | 7 |
| transferrin scFab 8D3 | |

In GD, currently available treatments including small molecules such as substrate reduction therapy or enzyme replacement therapy (ERT) fail to target GCase in the CNS compartment.

To overcome these limitations, we exploited the known ability of transferrin protein to cross the blood-brain barrier (BBB) through binding to the transferrin receptor (TfR), which transports the iron-binding protein transferrin into the brain 15.16. Moreover, we reasoned that hijacking this TfR-mediated pathway could also lead to increased lysosomal localisation of a cargo protein such as GCase 17.18. With this in mind, we fused a fragment of a transferrin receptor (TfR) antibody to recombinant human or murine GCase to generate a fusion protein we termed the GCase Brain Shuttle (GCase-BS). We assessed its efficacy in correcting GBA1-associated molecular changes in vitro and in vivo. We demonstrated that GCase-BS TfR-binders not only mediate successful transcytosis of GCase across endothelial cells of the BBB, but they are significantly more efficient than conventional ERT using recombinant GCase in terms of delivering enzyme to lysosomal compartment and driving the hydrolysis of patholog-ically accumulated lysosomal lipids in multiple neuronal models. Our results also uncover GD-associated lysosomal protein- and lipid defects that are rapidly corrected within the orga-nelle upon delivery of GCase-BS. Our data provide a pre-clinical proof of concept support for the use of GCase-BS for the treatment of GBA1-associated neurological dysfunction. Furthermore, this work provides insights into putative GD-associated lysosomal biomarkers downstream of GCase and highlights areas requiring further optimization. Given that the TfR targeting is so efficacious, it is conceivable that this approach may extend to multiple LSDs even in peripheral tissue that is not well targeted where significant unmet medical needs remain.

The AAV virus particles of the present invention overcome limitations in AAV bio distribution of current CNS AAV gene therapy approaches using Gcase as transgene. Currently available AAV capsids targeting the CNS transduce only a fraction of neurons leading to a low expression of the transgene in the target tissue. The use of a fusion protein of the present invention in an AAV gene therapy for the CNS enables cross-correction i.e. the fusion protein of the present invention is secreted by AAV virus particle transduced cells and the secreted fusion protein of the present invention is taken up via the TfR by non-transduced cells. This approach results in a larger number of cells with functional GCase and potentially higher efficiency and overcomes the limitation of AAV bio distribution of a traditional CNS gene therapy approach using Gcase as transgen. Therefore, the fusion protein of the present invention (GCase-TfR binder) can be either used as recombinant fusion protein or directly expressed in vivo from an AAV format.

Purified GCase-BS Molecules are Functional with Respects to Enzymatic Activity, Stability and TfR Binding

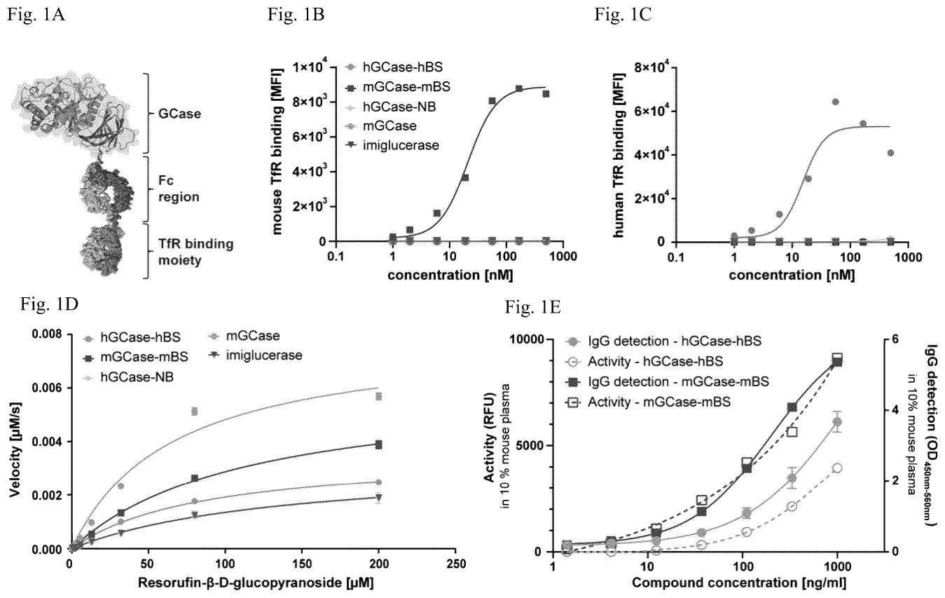

We designed GCase-BS molecules in which one chain of a human IgG1 Fc portion was fused to the C-terminus of GCase whereas the other chain of the Fc portion was fused N-terminally to an anti-mouse or anti-human TfR binding Fab. These fusion constructs, referred to as mGCase-mBS or hGCase-hBS, respectively, were designed using knob-into-hole technology (see 19; FIG. 1A), which was used to enable monovalent binding to TfR.

We generated a variety of insect cell (S2)-derived constructs and analysed the purified molecules to assess their TfR binding and enzymatic properties (FIG. 1B-D). Binding to mouse or human TfR was evaluated by FACS performed using suitable TfR-expressing cell lines, and the EC50 values of the selected fusion constructs were 21 nM for mGCase-mBS or 15 nM for hGCase-hBS, respectively (FIGS. 1B and C). We benchmarked the enzymatic properties of all molecules described in the manuscript to the conventional ERT molecule imiglucerase (commercially available as Cerezyme). There is a tendency for the Brain Shuttle constructs to be more efficient in terms of the ratio of turnover to affinity to resorufin-β-D-glucopyranoside. Naked GCase constructs and TfR non-binder (GCase-NB) are very similar to imiglucerase (FIG. 1C). In addition, initial testing by spiking the produced murine and human GCase-BS constructs into 10% mouse plasma for a period of 15 minutes and both stability and enzymatic activity were maintained (FIG. 1E).

### The TfR-Binding Module Improves Lysosomal Targeting and Substrate Reduction In Vitro

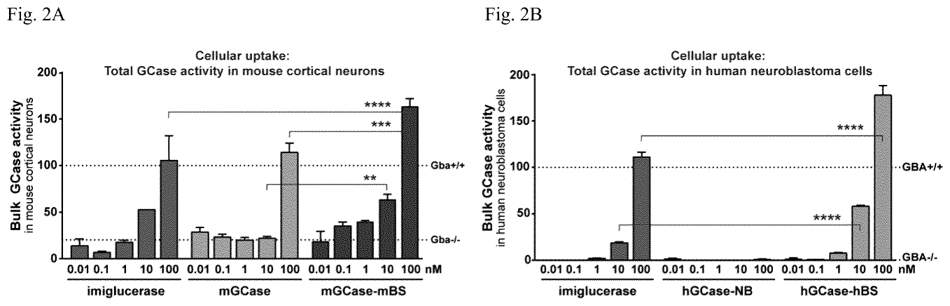

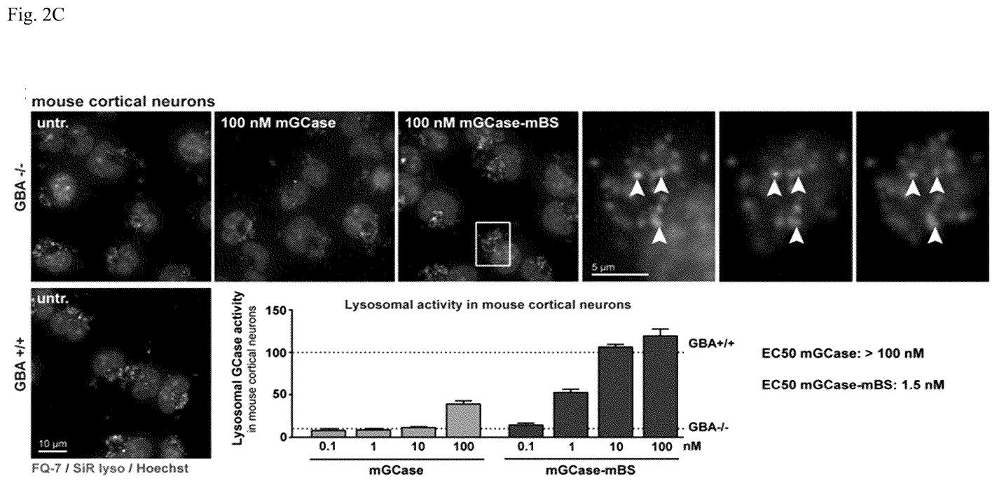

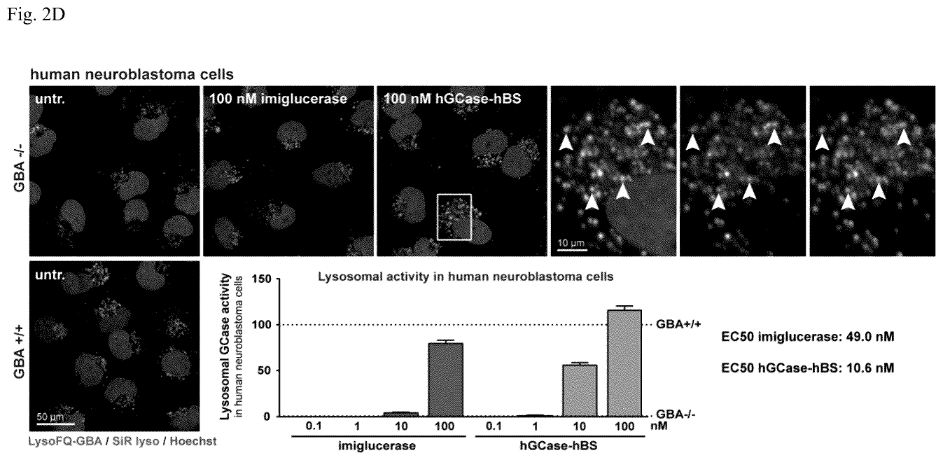

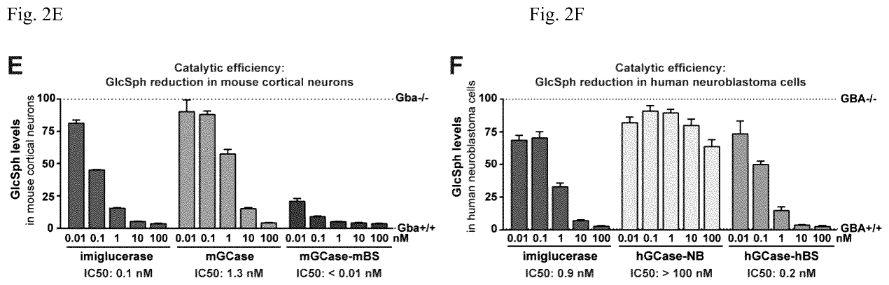

To investigate the effects of the TfR-binding module on cellular uptake and lysosomal efficacy, we used a variety of GCase-deficient cell lines: immortalised mouse cortical neurons from embryonic null allele Gba−/− mice 20, human pluripotent stem cell-derived neurons or human neuroblastoma cells (H4 cells) in which GBA1 was deleted (GBA−/−)²¹ or primary murine neurons harbouring a human GBA1 homozygous mutation (Gbal D409V/D409V)²². Both murine and human cell lines exhibit reduced basal GCase activity as well as significantly elevated lysosomal glycolipid levels compared to respective WT cells (Westbroek et al., 2016 and see FIG. 2 GBA+/+vs. GBA−/−).

In cellular uptake experiments, a 2 h treatment of immortalised mouse cortical neurons with mGCase-mBS led to a dose-dependent increase in total GCase activity which normalised function between 10-100 nM with respect to WT levels. Compared to mGCase or imiglucerase, mGCase-mBS showed a clear increase in cellular uptake most notable at the highest dose (FIG. 2A). Consistently also for the human molecule, in GBA1 KO H4 cells, a 2 h treatment with hGCase-hBS increased total GCase activity up to 3-fold more than imiglucerase, suggesting that uptake of hGCase-hBS is superior to GCase alone (FIG. 2B). Interestingly, there was less uptake of the control constructs, mGCase and hGCase fused to a TIR non-binder (FIG. 2B, hGCase-NB), illustrating that the in-house produced GCase constructs had reduced propensity for cellular uptake and lysosomal retention compared to the commercially available enzyme.

This could be explained by a different N-glycosylation pattern on the enzyme surface which is known to be involved in cellular uptake and intracellular trafficking 2.3.

We next exploited the novel GCase-specific fluorescence-quenched substrate LysoFQ-GBA (manuscript in review) to measure increase in lysosomal GCase activity in neurons. Cell imaging revealed that treating Gba-deficient murine cortical neurons for 2 h with mGCase or mGCase-mBS could restore lysosomal GCase activity to WT levels. We found that LysoFQ-GBA signal colocalized with the lysosomal probe SiR-lysosome and quantification of GCase activity showed that mGCase-mBS (EC50)=1.5 nM) was about 100-times more effective than mGCase (EC50)>100 nM) in restoring lysosomal GCase activity (FIG. 2C). In primary neurons derived from Gbal D409V/D409V mice, GCase activity was ˜7-fold higher for mGCase-mBS as compared to an equimolar dose of mGCase (32 nM, Suppl. FIG. 1C). In GBA1 KO H4 cells, a 2 h treatment with imiglucerase or hGCase-hBS led to a significant increase in lysosomal GCase activity. Quantification of the LysoFQ-GBA signal revealed that hGCase-hBS (EC50)=10.6 nM) was 5-fold more efficient at increasing lysosomal GCase activity as compared to imiglucerase (EC50)=49 nM) (FIG. 2D). These data illustrate that imiglucerase is less efficiently taken up into lysosomes than our hGCase-hBS construct, which actively engages with the TfR.

Upon analysis of the GD pathological lipid glucosylsphingosine (GlcSph) levels in both human and murine lines 48 h after treatment with their respective molecules we found that, in both cellular systems, lysosomal hydrolysis was significantly increased when GCase is fused to a TfR-binding moiety. Comparison of IC50 values revealed that mGCase-mBS is >100-fold more efficacious than mGCase alone and hGCase-hBS is ˜5-fold more efficacious than imiglucerase (FIGS. 2E and 2F). As a control, we showed that hGCase fused to a TfR non-binding module (hGCase-NB) resulted in limited substrate reduction, consistent with its inefficient cellular uptake (FIGS. 2B and 2F).

Using immortalised mouse Gbal KO cortical neurons or GBA1 KO H4 cells, we performed kinetic studies of GlcSph levels, whereby cells were incubated for 2 hours with varying concentrations of mGCase-mBS and hGCase-hBS, respectively, followed by washout, after which the kinetics of GlcSph hydrolysis were monitored (Suppl. FIGS. 1A and B). For both constructs in their respective cell lines, within 6 h after washout GlcSph levels were halved and at all concentrations tested reached maximal efficacy by 24 h. Interestingly, the dose-response relationship impacted the duration of treatment, whereby a short incubation of 100 nM and 10 nM of hGCase-hBS resulted in sustained lipid reduction over the 72 h course of the experiment whilst the 1 nM incubation showed glycolipid levels starting to rebound back to those seen in the disease state. These data suggest a 2 hours incubation sufficient to to resolve substrate levels over an extended period of time, suggesting a mechanism driven by the maximum concentration of GCase in lysosomes. These data provided the foundation of the in vivo pharmacokinetics and -dynamics (PK/PD) studies.

In hiPSC-derived macrophages from a healthy donor (GBA+/+) or from a PD individual with GBA1 genotype N370S/+, 9 days of treatment with 100 nM of hGCase-hBS led to a significant increase in total GCase activity (+58% in GBA+/+: +66% in GBA1 N370S/+. Analysis of GlcSph levels showed that its levels are significantly elevated in the GBA1 N370S/+ line (+88%) and could be substantially reduced upon treatment with hGCase-hBS (55% reduction at 100 nM, 29% reduction at 10 nM). These data suggest that the construct is functional in macrophages and normalises enzyme activity and GlcSph lipids in a cellular model harbouring a GD/PD pathological mutation.

In human pluripotent stem cell-derived GBA1 KO dopaminergic neurons²⁴, treatment with 10 nM of hGCase-hBS efficiently normalised GlcSph levels, suggesting that in human midbrain neurons, relevant for PD and GD hGCase-hBS is efficacious at low concentrations.

### GCase-BS Lysosomal Mode of Action In Vitro

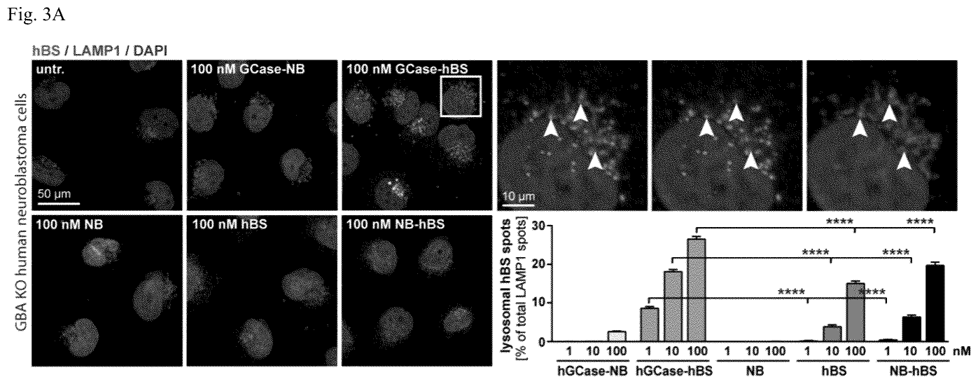

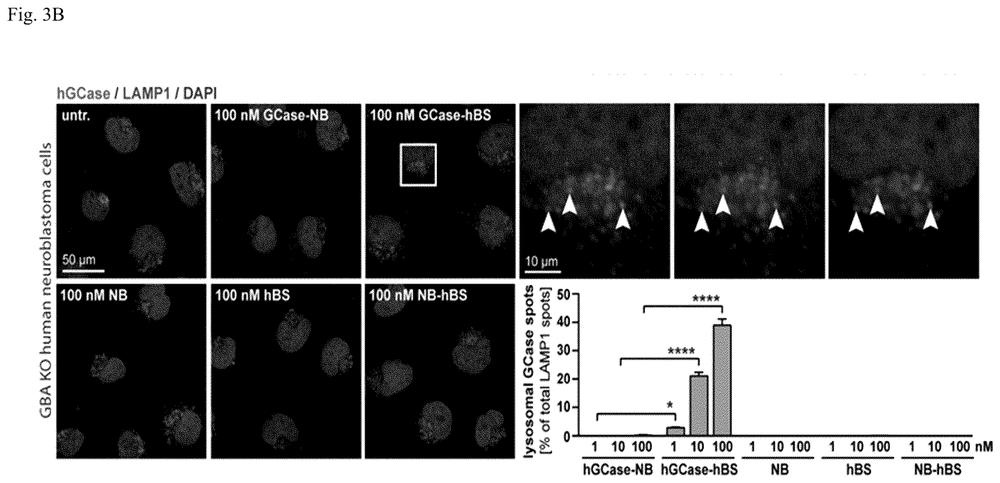

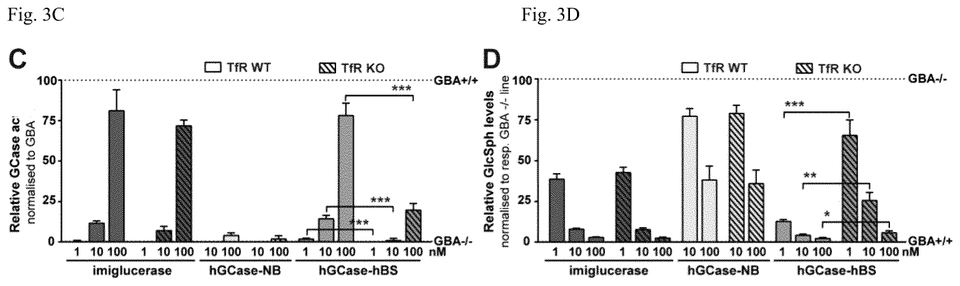

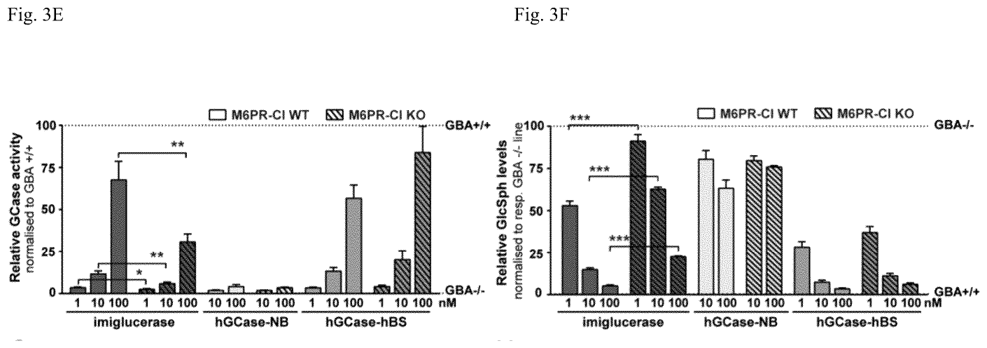

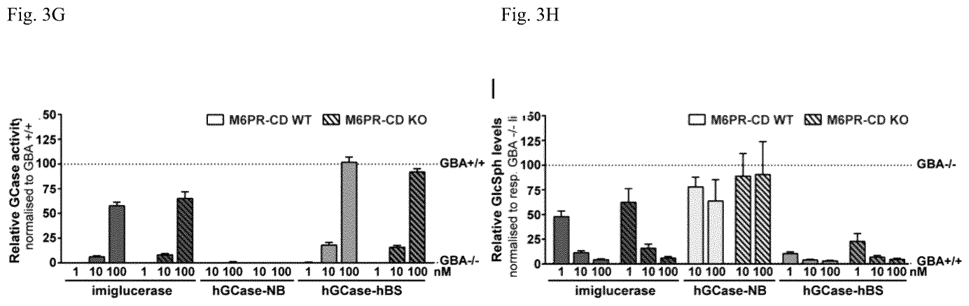

It is well established that TfR binders transport therapeutic molecules across the BBB ^{18,25,26}. However, it is unclear how the BS module of GCase-BS helps to facilitate CNS cellular uptake and lysosomal targeting of GCase. Inspired by the findings that the BS modules increased both lysosomal exposure and efficacy for hydrolysing GlcSph within lysosomes, we aimed to elucidate the underlying mechanisms in more detail. To this end, we compared the ability of four constructs to target the lysosome: 1) GCase attached to a TfR-binding molecule (hGCase-hBS), 2) GCase attached to a TfR-non binder (hGCase-NB), 3) an antibody cargo (NB-hBS) and 4) the BS (hBS) and control BS(NB) moieties alone. GCase-deficient H4 cells were incubated with the molecules for 2 h and the localisation of both the Brain Shuttle moiety and enzyme were monitored. We found that there was negligible cellular uptake of hGCase-NB and exposure to the lysosome, which we assessed based on little to no IgG or hGCase cellular immunoreactivity colocalizing with LAMPI (FIGS. 3A and 3B). This observation was in line with the previous findings that hGCase-NB only led to very limited cellular uptake and lysosomal lipid reduction (FIGS. 2B and 2F). We also observed that 2 h treatment with hGCase-hBS led to colocalization of both GCase (up to 50% colocalized GCase spots at 100 nM) and hBS (up to 25% colocalized hIgG spots at 100 nM) with LAMPI. We observed considerably less colocalization for NB-hBS or hBS alone (FIGS. 3A and 3B), suggesting that efficient lysosomal targeting required a combination of both the BS module and its GCase cargo.

To determine which receptors are required by hGCase-hBS for both cellular uptake and lysosomal targeting/efficacy, we generated several double KO H4 lines lacking GCase and either TfR or cation-dependent M6PR (M6PR-CD) or cation-independent M6PR (M6PR-CI) to furnish us with GBA/M6PR-CD KO. GBA/M6PR-CI KO, and GBA/TIR KO cells. After a 2 h treatment with imiglucerase, hGCase-NB, or hGCase-hBS we observed that absence of TfR resulted in marked reduction of both cellular uptake of the construct and significant impairment in the ability of the construct to reduce GlcSph. In contrast, for imiglucerase, cellular uptake and efficacy remained unaffected (FIGS. 3C and 3D). However, using the M6PR-CI KO we found both the uptake of enzyme and its ability to reduce lysosomal glycolipids were greatly impacted for imiglucerase, whereas the activity of hGCase-hBS was unaffected (FIGS. 3E and 3F). Interestingly, we found that M6PR-CD played no role in neither cellular uptake nor lysosomal lipid reduction for any of the molecules (FIGS. 3G and 3H).

Collectively, our data suggest that, as expected, imiglucerase uses the M6PR-CI for cellular and lysosomal uptake whereas the GCase-BS constructs predominantly access the lysosome by engagement and sorting through interaction with the TfR.

### GCase-BS Corrects Lysosomal Phenotypes

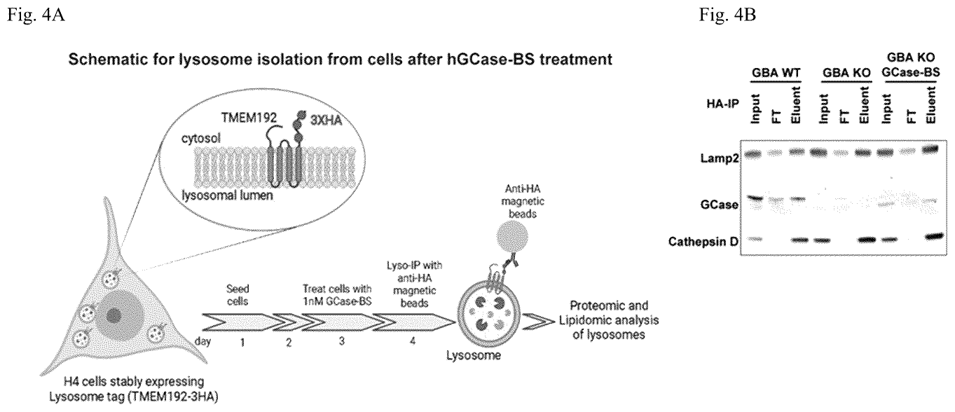

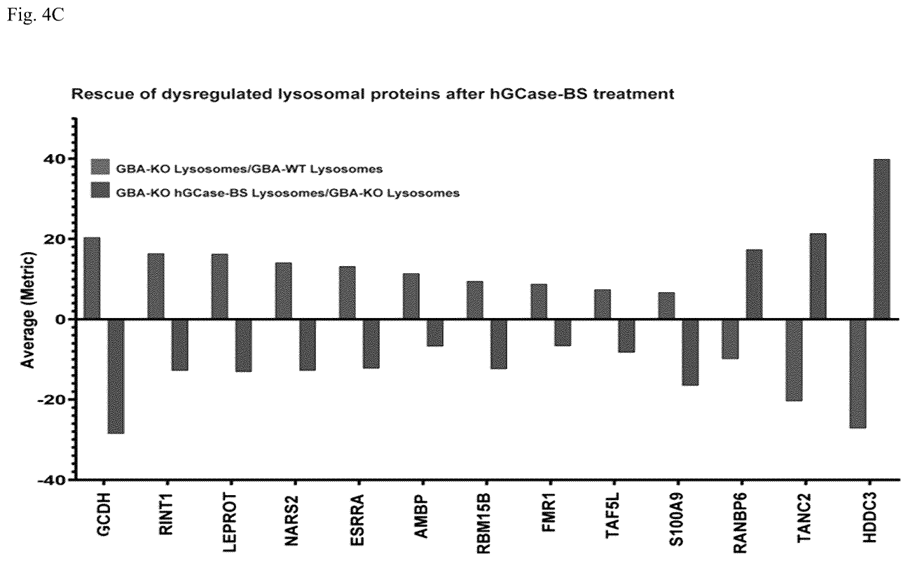

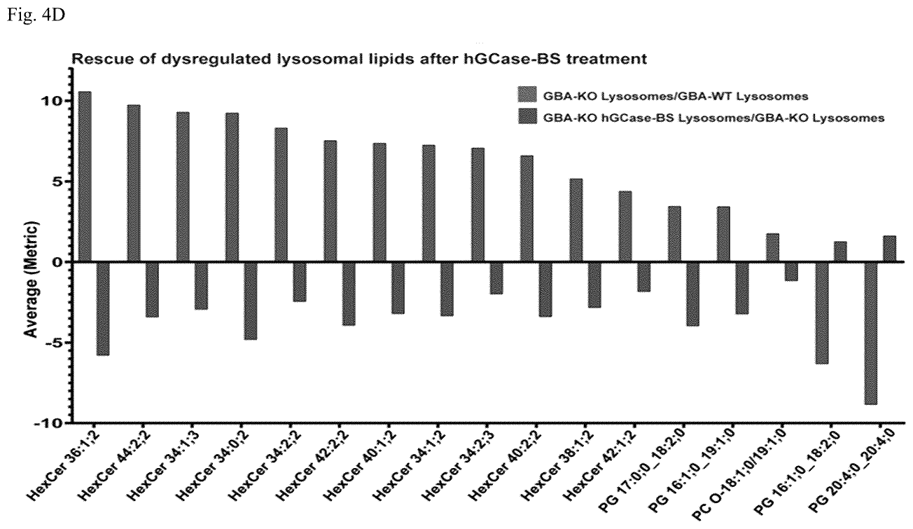

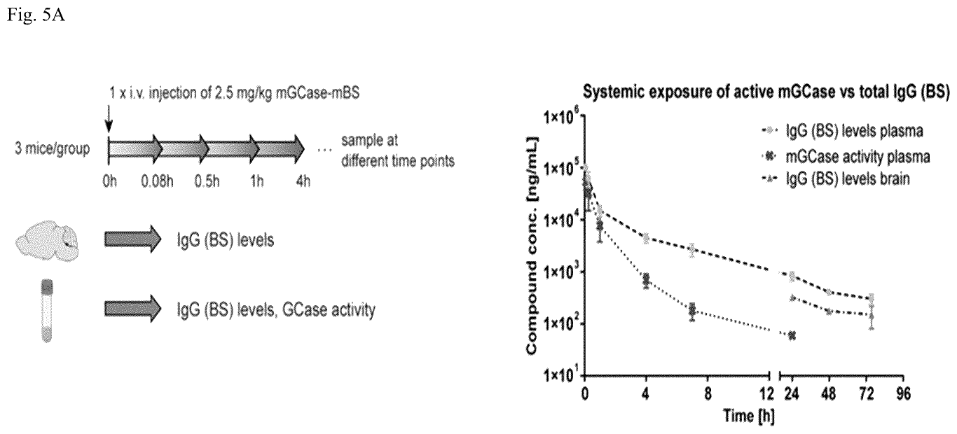

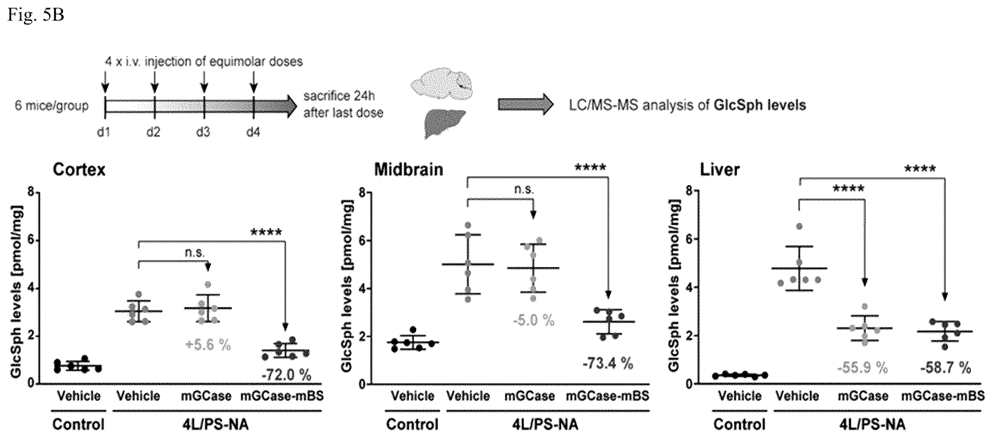

Having demonstrated the efficacy of the Brain Shuttle in delivering GCase to lysosomes in neuronal cell lines, we sought to investigate the consequences of reinstating lysosomal GCase on the molecular architecture of lysosomes. Since treatment of GBA KO cells with 1 nM hGCase-hBS was sufficient to normalise GlcSph levels without saturating the system, we used this condition to better understand the lysosome-specific changes in proteins and lipids. To this end, we established an experimental paradigm to specifically enrich and profile lysosomes for proteomic and lipidomic perturbations upon hGCase treatment (FIG. 4A). We used a method to purify lysosomes from cells that relies on stable expression of the so-called Lysosome-tag, TMEM192-3XHA 27. Expression of the Lysosome-tag facing the cytosolic side makes it pos-sible to rapidly and efficiently immunoprecipitate lysosomes using anti-HA antibodies after cell lysis (FIG. 4A). Following treatment of cells with hGCase-hBS, we purified lysosomes in this way and confirmed they were intact by probing the presence of the lysosomal membrane protein. Lamp2 and soluble lysosomal lumen proteins. Cathepsin D and GCase (FIG. 4B. Eluent fraction). The isolated lysosomes, as well as whole-cell extracts, were subjected to global protein profiling using HRMTMID/ID+ mass spectrometry. Proteomic analyses showing enrichment of bona fide lysosomal proteins within the isolated lysosomal fraction, as compared to whole-cell extracts, further validated the isolation protocol delivering intact lysosomes (Suppl. FIG. 3). The list for bona fide lysosomal proteins includes proteins shown to be of lysosomal origin by comparative proteomic analysis of lysosomes from mammalian cells^{28,29}. Principal component analysis (PCA) of the proteomic dataset showed a clear separation between lysosomes and whole-cell extracts groups, as well as a clear separation between WT and GBA KO groups. Several proteins were significantly upregulated or downregulated in lysosomes and whole-cell extracts from H4 GBA KO cells as compared to those from H4 WT cells (FIG. 4C and Suppl. FIG. 5). Treatment of H4 GBA KO cells with 1 nM hGCase-hBS was sufficient to readjust the altered protein levels both in the lysosomes (FIG. 4C) and whole-cell extracts. One of the proteins that was significantly increased in GCase-deficient lysosomes is the pro-inflammatory mediator S100A9 (log 2FC-GBA KO lysosomes vs WT lysosomes: 1.77). Interestingly, S100A9 has been shown to co-localize and co-aggregate with alpha-synuclein in Lewy bodies in PD patients and in vitro studies suggest that S100A9 might alter the aggregation kinetics of alpha-synuclein 30.31 Treatment with hGCase-hBS could efficiently revert the observed increase in S100A9 levels in GCase-deficient lysosomes (log 2FC-GBA KO-hGCase lysosomes vs GBA KO lysosomes: −2.49). Another interesting protein that was significantly increased in GCase-deficient lysosomes and efficiently reverted by hGCase-hBS treatment is Estrogen related receptor alpha (ESRRA) (log 2FC-GBA KO lysosomes vs WT lysosomes: 3.27: log 2FC-GBA KO-hGCase lysosomes vs GBA KO lysosomes: −3.17). ESRRA has been shown to increase the cellular expression of monoamine oxidases (MOA)—a mitochondrial enzyme responsible for oxidation of dopamine. Moreover, parkin was shown to negatively regulate this process by ubiquitination and degradation of ESRRA 32.

Using the same experimental paradigm employed in the proteomic study, we performed mass spectrometry-based shotgun lipidomic analysis of lysosomes and whole-cell extracts to assess the impact of hGCase-hBS on the lipid profile in GCase-deficient H4 cells. Many lipid species belonging to the hexosylceramide (HexCer) family are increased in lysosomes and whole-cell extracts lacking GCase and are efficiently reverted towards basal levels with 1 nM of hGCase-hBS (FIG. 4D). Our data also supports that in addition to GlcSph, GCase catalyses the breakdown of several species of HexCer that differ in chain length, double bonds, and hy-droxylation. We also found that levels of lipid species belonging to the phosphatidylcholine (PC), phosphatidylglycerol (PG) and phosphatidylserine (PS) were significantly altered in GCase-deficient cells (FIG. 4D). Notably, several of these altered lipid species were subsequently corrected to basal levels upon treatment with hGCase-hBS (FIG. 4D). Taken together, our data suggests that GCase deficiency in cells leads to global lipid changes that can be corrected in a rapid time frame after incubation of hGCase-hBS.

### GCase-BS Proof of Concept In Vivo

We next performed a single-dose study using C57BL/6 mice in which mGCase-mBS levels were analysed measuring both the BS moiety (total IgG) of the construct by an IgG immuno-assay and GCase enzymatic activity using a chemical substrate. In this way we could assess the pharmacokinetics (PK) and stability of the molecule in vivo. The mGCase-mBS construct shows a high systemic clearance (>30 ml/h/kg). Initially, plasma levels measured with both assays were similar (5 min after injection), while for the entire period of observation, the exposure in terms of area under the curve (AUC) was approximately ¼ lower when enzymatic activity was measured as compared to the exposure measured using the IgG immune-assay (FIG. 5A). This suggests that the stability of the GCase domain was affected in the blood over time. As the uptake into the brain is the rate limiting step, assessing brain exposure by measuring total IgG levels from brain lysates showed a slightly lower but parallel pharmacokinetic profile (Cmax for total IgG in brain at 24 h: 2.1 nM). No active mGCase could be detected in plasma after 24 h. likely due to lack of sensitivity of the GCase enzymatic activity assay (LOQ=0.4 nM).

To demonstrate proof of concept for the GCase-BS construct in vivo, initially we performed a multiple dose (4 doses of 2.5 mg/kg) study in a mouse model of GD which is named 4L/PS-NA. These mice have a homozygous Gbal mutation (Gba V394L/V394L) and a prosaposin KO(Psap−/−). The mice exhibit neuronal phenotypes that are similar to those in GD2 or GD3 patients, eg. decreased GCase activity and a strong accumulation of GlcCer and GlcSph in the lysosomal compartment 33.34. Assuming that the pharmacokinetics in the brain in these mice also run in parallel to the blood profile, the rationale for the dosing regime was to generate high enough brain exposures to allow target engagement and so induce a clear pharmacody-namic effect on relevant markers. We found that at baseline, compared to control littermates, the 4L/PS-NA mice showed an elevation of GlcSph levels to 4-fold (cortex), 3-fold (midbrain) or even 13-fold (liver). 24 h after the last dose of mGCase-mBS in the 4L/PS-NA mice, lipid levels in both the cortex and midbrain were reduced by ˜72% (FIG. 5B). Non-conjugated mGCase alone was not effective at reducing lipids in multiple brain regions suggesting that the BS module is key for reducing brain lysosomal GlcSph (FIG. 5B). In the liver, however, both mGCase and mGCase-mBS resulted in ˜60% normalisation of substrates suggesting they are similarly potent in the liver which was unexpected based on the observation that TFR is more efficient (FIG. 5B). This unexpected observation might be explained by differential expression patterns of M6PR and TfR in this tissue compared to brain tissue including almost no TFR in hepatocytes. Similar results were obtained in mice carrying only the Gbal mutation (Gba V394L/V394L) alone. In summary, these results suggest that the Brain Shuttle module not only increases lipid reduction potential but also promotes crossing of the BBB into the brain parenchyma in two Gbal murine models.

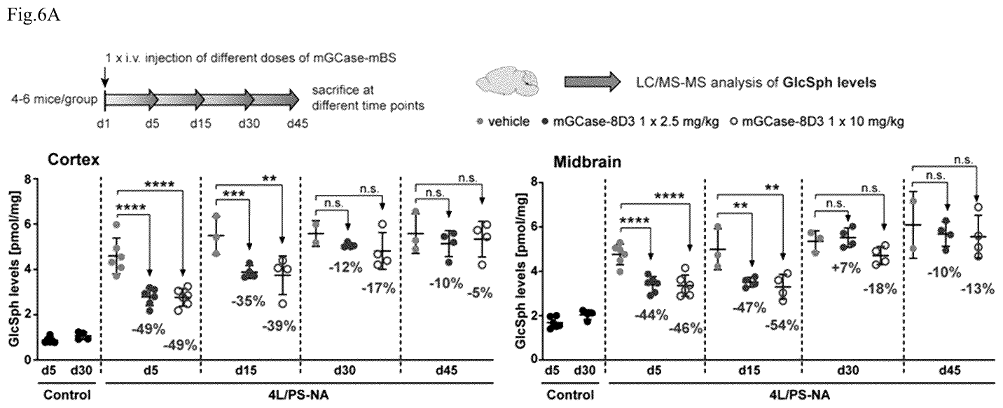

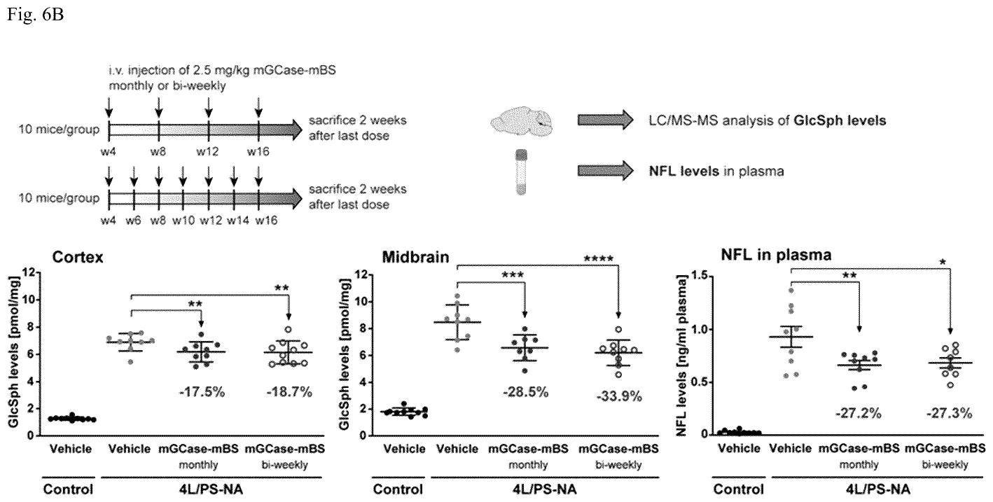

Subsequent single dose dose-response experiments were performed at doses ranging from 0.2 mg/kg to 2.5 mg/kg and results suggested that doses below 2.5 mg/kg are not sufficient to significantly lower brain lysosomal lipid levels. Therefore, to gain more insight into suitable dosing frequencies and the trajectories of pathological lipid rebound after last dosing, we injected a single-dose of either 2.5 mg/kg or 10 mg/kg and analysed tissue at various time points after the last dose in 4L/PS-NA mice (4-6 mice per group). GlcSph analysis revealed that both doses similarly led to ˜50% reduction of substrate as benchmarked to control animals at day 5 post injection. The kinetics of changes of GlcSph levels revealed that at 15 days post injection, GlcSph levels were still significantly lower in both cortex and midbrain and that it took up to 45 days to return to levels of GlcSph seen in untreated animals. These results suggest that lipid reduction after treatment is sustained and a bi-weekly or monthly dosing frequency could be sufficient to reach beneficial effects (FIG. 6A).

To test whether this was correct, we performed a multi-dose chronic study, where we injected 4L/PS-NA mice monthly or bi-weekly with 2.5 mg/kg of mGCase-mBS starting from 1 month of age for 3 months. In this study, we monitored GlcSph levels in the brain and, as a biomarker of neurodegeneration, NFL levels in plasma (biomarker of neurodegeneration previ-ously reported to be elevated in this model³³. Two weeks after the last injection, in both cortex and midbrain, there was an ˜18% and ˜30% reduction of substrate levels in cortex and midbrain, respectively (FIG. 6B). We did not observe a marked difference between the bi-weekly and monthly dosing regimens. Notably, NFL levels were 35-fold elevated in the 4LPS-NA mice plasma compared to controls. Using both dosing regimens we observed significant reductions to almost 30% of untreated 4LPS-NA. These results suggest that both bi-weekly or monthly dosing is adequate to lower lipids sufficiently to alter the trajectory of neurodegeneration in an aggressive GD model (FIG. 6B).

### DISCUSSION

The classical view of the lysosome is as a terminal catabolic station that relieves cells of waste products³⁵. This view has recently been expanded by the discovery of new roles for the lysosome in nutrient sensing, transcriptional regulation, and metabolic homeostasis 35. At the subcellular level, LSDs manifest in abnormal intra-lysosomal accumulation of metabolites due to defects in one or multiple catabolic pathways caused by genetic defects that lead to reduced levels of lysosomal enzymes 36. The clinical manifestations of LSDs vary widely but neurological symptoms are common features 37. Restoring the levels of the missing enzyme is a highly effective treatment and is standard of care in many different types of LSDs. However, the recombinant enzymes used for ERT lack the ability to cross the BBB. This leads to no or poor brain exposure of therapeutic enzymes and subsequent failure in reversing neurological complications in patients.

The neuronopathic forms of GD (GD2 and GD3) and GBA-PD are devastating neurological diseases manifesting as a result of GCase lysosomal dysfunction culminating in neurodegeneration. There is a compelling body of preclinical and clinical data suggesting that increasing GCase activity in the brain can revert the underlying lysosomal dysfunction, thus having a marked impact on disease trajectory 38. Collectively, our data suggests that fusion proteins comprising GCase and TfR binders represent a promising therapeutic approach for GBA1-associated neurological diseases. Current ERT for GCase, which is an effective treatment for GD1, has various limitations that could be addressed to optimise efficacy for CNS potency as reflected in markers including pathological lysosomal lipid reduction, rapid clearance from blood, and stability of the enzyme after infusion. The GCase-BS construct therefore breaks new ground by offering significant potential advantages. We show that it not only passes the BBB endothelium, but also results in 5-100 times more efficient glycolipid reduction in multiple relevant human cellular systems (FIG. 2).

Recently it has been shown both preclinically and in human clinical trials that the lysosomal enzyme iduronate 2-sulfatase (IDS) coupled to TfR binders are able to pass the BBB by demonstration of CNS target engagement (eg. CSF/NFL) 39.40. This effort has culminated in the approval of JR-141 (IDS TfR binder) for mucopolysaccharidosis (MPS) type I for central disease manifestations in Japan 41. Recent work has also highlighted that this approach extends to other lysosomal proteins including progranulin. Since the concept of TfR binding to pass the BBB to treat MPS2 and neuronal ceroid lipofuscinosis (NCL) has been validated in humans 42.43, we now extend these findings for GBA1-related neurodegenerative diseases.

Soluble and membrane-bound lysosomal proteins have sorting signals that are recog-nized by sorting receptors for their proper delivery to the endolysosomal system through various trafficking routes. GCase, as a lysosomal enzyme, takes a rather unique path to the lysosome involving the lysosomal integral membrane protein 2 (LIMP2) receptor⁴⁴. The sorting signals of soluble lysosomal proteins can either be folded polypeptide sequences displayed on the protein surface or specific glycan modifications. Modification by mannose 6-phosphate (M6P) is a well-characterised sorting signal, and for GCase ERT, the glycan is tailored to make the “mechanism of action” dependent on this pathway. Once tagged with M6P residues, enzymes are routed to the lysosome by interaction with the cation-independent mannose 6-phosphate receptor (M6PR-CI, also IGFR2). Since the M6PR-CI is not only localized at the trans-Golgi network and endosomes but also at the plasma membrane^{45,46}, exogenously delivered GCase (ERT) can be retrieved by M6PR-CI and sorted to the endolysosomal system, and subsequently to the lysosome. This mechanism represents the foundation of effective ERT and is used by the commercially available forms of GCase, including the enzyme imiglucerase.

In the case of GCase-BS, we demonstrated that uptake, lysosomal exposure and hydrolysis of pathological GCase-targeted lipids are far more efficient and potent than the enzyme on its own. We show that this “mechanism of action” is solely dependent on the TfR and not the conventional receptor of ERT. M6PR. Notably, the GCase construct with an inactive TfR binder (hGCase-NB) displays no detectable uptake or substrate reduction indicating that this construct is unable to effectively use the M6P pathway through M6PR-CI sorting (FIG. 3). This suggests that hGCase-NB either lacks the necessary glycan modifications to engage with the M6PR-CI or the Brain Shuttle interferes with efficient uptake. However, when an active TR binder is used as in the GCase-BS construct, strong cellular uptake and robust substrate reduction is seen. Thus, in the neuronal cells used in this study, the TfR provides an efficient pathway into lysosomes. Importantly, at the BBB this TfR sorting pathway is very likely different. Here, the Brain Shuttle construct is transported across the BBB into the brain parenchyma if the engagement with the TfR possesses certain features, including a monovalent binding-mode that appears to prevent lysosomal sorting within the endothelial cells at the BBB. Taken together, data pre-sented in this work shows that the TfR pathway can provide both productive transport across cells at the BBB and subsequently enhanced uptake and efficacy in hydrolysis of lysosomal glycolipids in neuronal cells within the brain. It is also important to appreciate that the cargo associated with the BS in a fusion construct may influence trafficking of the construct. For example, if the cargo is a high-affinity antibody against a specific target in the brain, it is likely that the BS construct will be directed toward this target due to the stronger binding compared to the binding to the TfR. Therefore, whether other lysosomal enzymes coupled to TfR binders share features seen here for GCase-BS, including increased uptake and efficacy, remains to be determined.

Upon systemic administration of mGCase-mBS rapid clearance of the construct from blood is observed (FIG. 4A). Notably. GCase enzymatic activity seems to decline in blood much faster than the shuttle domain of the construct. Only about 0.1% of the injected enzymatic activity is detectable in circulation after 24 h. This suggests that the GCase enzyme is much less stable in the blood in vivo than the Brain Shuttle and is likely driving the clearance of the construct from the peripheral compartment. This observation highlights current limitations that could be further optimised, including clearance and enzyme stability. Since it was clear that the molecule loses stability over time in the blood, one could make attempts to stabilise GCase. For example, there are reports that GCase mutants may have increased stability 47.48 The short half-life and stability of the GCase-BS construct likely limit efficacy. The combination of pharma-cological chaperones with ERT has shown the potential to improve the bioavailability of another recombinant human GCase enzyme in preclinical research and clinical trials⁴⁹⁻⁵². This strategy could be applied to GCase-BS using a known GCase chaperone such as isofagomine. Such an approach could stabilise the GCase-BS enabling more active enzyme to pass the BBB. Since uptake of the molecule is solely dependent on TR binding, it is therefore independent of terminal mannose residues suggesting that tailoring the glycans on the construct could increase stability and bioavailability. For example, the negative charge of sialic acids is thought to prevent aggregation by creating a repulsive force between therapeutic molecules and preventing kidney filtration⁵³. Accordingly, polysialylation of therapeutics has been shown to significantly increase half-life^{54,55}. Future experiments will be required to test these concepts.

Mouse models of Gbal-associated PD have limitations that impact their utility for the purpose of studying disease biology. Such models, for example, have very little accumulation of lipids in the brain 56. We therefore leveraged the 4LPS/NA model that harbours a homozygous knock-in of Gba V394L/V394L and has only one allele of psap. This mouse model has extensiveaccumulation of brain lipids and was ideal for the purpose of monitoring pharmaco-dynamic effects on lipids such as GlcSph. We showed that with a single dose of mGCase-mBS GlcSph levels are reduced by close to 50% relative to WT levels, while four doses spaced 24 hours apart (loading phase) resulted in about a 70% reduction in relation to WT levels. These results imply mechanistically that while the brain in vivo effect is in part driven by the maximum concentration in blood (Cmax), supported by the in vitro work showing that lysosomal hydrolysis is fast, the capacity of the TfR at the BBB to shuttle mGCase-mBS can become saturated. This view is supported by observations showing that doses from 1-10 mg/kg result in a limited dose-response relationship.

Within the liver, both the free GCase enzyme and the mGCase-mBS construct are highly active, verifying that the GCase ERT is functional and active in vivo outside the brain yet cannot penetrate into the CNS. Similar efficacies of both mGCase and mGCase-mBS in liver could be explained by different expression levels of M6PR and TfR in this tissue compared to brain cells. Another notable observation is the marked and sustained reduction in GlcSph levels in the brain after ending treatment. Both in cortex and midbrain, a significant reduction of GlcSph is observed up to 15 days post treatment and clear trends even up to 45 days were seen (FIG. 4C). Recently it has been shown that the NF-L levels in the CSF and plasma in this 4L/PS-NA GD mouse model are 9-fold higher in plasma and 70-fold elevated in the CSF compared to wild-type controls⁵⁷. Similar increases in plasma NF-L was observed in this study (FIG. 4D). 12-Weeks treatment with the mGCase-mBS construct, using a dosing frequency that is likely acceptable for delivery of human therapies to treat GD or GBA-PD (bi-weekly/monthly), was able to significantly reduce these abnormal NF-L levels. Since NF-L is found in large myelinated axons of neurons, this measurable therapeutic effect likely originates within the CNS. The approximately 30% reduction of NFL to WT levels is striking since the model has a component of neurodegeneration that is independent of GCase, driven by PSAP haploinsufficiency whereby PSAP influences other lysosomal functions such as progranulin metabolism⁵⁸.

CSF GlcSph is a putative translational biomarker for both GD and GBA-PD that is downstream to elevating GCase in brain. However, as highlighted by the recent venglustat clinical trial, reductions of these key pathological lipids will be not sufficient to determine whether GBA1-dependent lysosomal homeostasis has been restored^{59,60}. To that end, we monitored protein and lipid changes in both whole cell lysates and rapidly purified lysosomes from a GD cell model. We identified both key proteins and lipids that were abnormally regulated in GD cells and rapidly reverted after addition of hGCase-hBS. Future work could address the role of these molecular changes on GBA1-assocoated neurodegeneration and ultimately may serve as proximal GBA1 pathway lysosomal biomarkers downstream of target engagement. A key translational biomarker may require rapid lysosomal purification of peripheral cells to demonstrate functionality in the lysosomal compartment³⁵.

In summary this work suggests that GCase-TfR binder fusion proteins can correct lysosomal deficiencies and hence represent an attractive therapeutic avenue for GBA1-associated neurodegeneration. We furthermore propose opportunities for future optimization of such constructs and provide the foundation for potential biomarkers that can be employed to examine efficacy of lysosome-targeted therapeutics in a translational context.

### Methods

### Reagents and Antibodies

Chemicals were purchased from Sigma Aldrich, if not stated otherwise. Antibodies used to perform immuno-based experiments were: rb mAb to hGCase (Abcam, #ab128879), rb mAb to TFRC (Abcam, #ab214039), rb mAb to M6PR cation-independent (Abcam, #ab124767), rb mAb to M6PR cation-dependent (Abcam, #ab134153), HRP-conjugated rb pAb to GAPDH (Abcam, #ab9385), ms mAb to Lamp2 (Thermo Fisher. #MA1-205), rb mAb to Cathepsin D (Abcam, #ab75852),

### Cell Culture

Human neuroglioma cells (H4) were maintained in DMEM/F-12 (#11039-021) supplemented with 10% fetal bovine serum (#A31605-01) and penicillin (100 U/ml), streptomycin (100 μg/ml) (ThermoFisher).

Human neurons were differentiated from neural stem cells (NSCs) for 6 weeks in differentiation media (DMEM/F-12 with GlutaMax (#31331093) and neurobasal medium (#21103049) supplemented with 1× B27 (#12587010), 1× N2 (#17502048), 0.1% (v/v) beta-mercaptoetha-nol (#31350010), penicillin (100 U/ml), streptomycin (100 μg/ml), laminin ( 1/500) and cytokines: 20 ng/ml BDNF (Peprotech #450-02), 10 ng/ml GDNF (Peprotech #450-10), 100 μM ascorbic acid 2-phosphate (Sigma #A8960) and 500 μM cAMP (Sigma #D0627-5X1G).

Mouse immortalized primary neurons were maintained in neurobasal medium supplemented with 1× B27, 1× GlutaMax, penicillin (100 U/ml), streptomycin (100 μg/ml) and laminin ( 1/500).

All cells were kept at 37° C. in a humidified 5% CO2 atmosphere.

### Animals

For in vivo studies, we used the 4L/PS-NA mouse model resembling some neuronopathic phenotypes of GD.

These animals contain a V394L mutation in the GCase locus and a prosaposin gene knockout (Sun et al., 2005). Control littermates (WT for prosaposin gene) were used as a baseline.

Breeding and studies were carried out at QPS Austria according to respective animal handling regulations.

### Generation of H4 KO Cell Lines

CRISPR/Cas9 gene editing was performed to generate several KO H4 cell lines: In brief, H4 GBA KO cells were seeded at 1E5 cells/well into a 6 well-plate and transfected with RNPs at a 1.8:1 ratio (sgRNA: Cas9 nuclease) by lipofection. Lipofection reagents were purchased from Thermo Fisher Scientific (Lipofectamine™ CRISPRMAX™ Cas9 Transfec-tion Reagent #CMAX00015). Media was changed to full growth media after 18 h and cells were subjected to limiting dilution to obtain monoclonal cell populations. Loss of protein was confirmed by Western Blot.

| Synthetic | sgRNAs were purchased from Synthego: |
|---|---|
| 1) TfRC: | guide #1: G*U*G*AUCGUCUUUUUCUUGAU (Seq. Id.No. 8) |
| | guide #2: A*A*A*UGCUGACAAUAACACAA (Seq. Id.No. 9) |
| | guide #3: A*G*A*UGGCGAUAACAGUCAUG (Seq. Id.No. 10) |
| 2) M6PR-CD: | guide #1: A*A*U*CAACAAAAGUAAUGGGA (Seq. Id.No. 11) |
| | guide #2: U*U*C*AGGGUGUGCCGGGAAGC (Seq. Id.No. 12) |
| | guide #3: U*A*C*AGCUUUGAGAGCACUGU (Seq. Id.No. 13) |
| 3) M6PR-CI: | guide #1: U*U*G*AAUUGUGCAGGUAACGA (Seq. Id.No. 14) |
| | guide #2: C*G*U*GUCCCAUGUGAAGAAGU (Seq. Id.No. 15) |
| | guide #3: C*G*U*CGGUGGCACCGCAGAGG (Seq. Id.No. 16) |

Absence of respective proteins was determined by Western Blot analysis. In brief, cells were lysed in RIPA buffer, supplemented with protease inhibitors (Roche, Cat #: 11 873 580 001), and 10 μg of total protein were subjected to SDS-PAGE (Invitrogen NuPAGE system). Semi-dry transfer at 23 V for 6 min was used to transfer proteins onto a nitrocellulose membrane, which was blocked in 5% milk/TBST (Tris-buffered saline+0.05% Tween) and subsequently incubated with primary antibody at 1/1000 o/n at 4° C. After 3 washes in TBST, HRP-labelled secondary antibody was incubated for 1.5 h at RT at 1/10000 in 5% milk/TBST. Proteins were detected using the SuperSignal West Dura Extended Duration Substrate Kit (Thermo).

### Expression and Purification of Recombinant Human and Mouse GCase(-BS) Constructs

The recombinant hGCase and mGCase as well as hGCase-hBS and hGCase-NB were expressed in Schneider S2 cells (Drosophila cell line), using the expression vector pEx-preS2_1-A. hGCase and mGCase constructs were designed to express with a C-terminal His8-tag including a glycine-serine (GS) linker and a sortase recognition site (Sor). hGCase-hBS was designed by fusing one chain of a human IgG1 Fc portion devoid of Fcγ receptor binding to the C-terminus of human glucocerebrosidase and the other chain N-terminally to an anti-human TfR binding Fab using knob-into-hole technology. mGCase-mBS was expressed in two parts (mGCase-Sor and anti-mouse TfR binding Fab) and subsequently coupled by sort-ase-mediated site specific conjugation.

To purify mGCase or hGCase, cell supernatant was filtered and passed through a HiTrap Con A 4B column (GE Healthcare) using HiTrap Con A-Buffer (20 mM Tris/HCl at pH 7.4 with 0.5 M NaCl, 1 mM MnCl2, 1 mM CaCl2, 0.02% (v/v) NaN3 and 0.5 M Methyl α-D-mannopyranoside for elution). Eluted target protein was further purified with a HisTrap HP column (GE Healthcare) using HisTrap-Buffer (50 mM HEPES at pH 7.6 with 0.5 M NaCl, 0.02% NaN3 and 0.5 M imidazole for elution) followed by a hydrophobic interaction chromatography (HIC) with a Toyopearl Butyl-M 650 HIC column (Tosoh Bioscience) using HIC-Buffer (20 mM MES at pH 5.5 with 0.5 M KCl, 0.02% NaN3) for binding and 80% (v/v) ethylene glycol for elution.

To purify hGCase-hBS or hGCase-NB, cell supernatant was cleared by filtration and loaded onto a HiTrap Con A 4B column (GE Healthcare) as the first purification step. HiTrap Con A-Buffer (20 mM Tris/HCl at pH 7.4 with 0.5 M NaCl, 1 mM MnCl2, 1 mM CaCl2, 0.02% (v/v) NaN3 and 0.5 M Methyl α-D-mannopyranoside for elution) was used. Eluted target protein was further purified via a Capture Select KappaXL column (GE Healthcare) column using 25 mM Tris/HCl at pH 7.0, 25 mM NaCl, 5% (v/v) glycerol, 0.02% NaN3 as binding buffer. An additional wash step with binding buffer and 1% (w/v) CHAPS was in-cluded to remove endotoxins. The target protein was eluted with 20 mM citric acid at pH 3.5, 0.1 M glycine, 5% (v/v) glycerol and 0.02% NaN3 from the column. The pH was adjusted directly after protein elution to pH 6.0.

All purified constructs were finally dialysed against a slightly acidic solution (20 mM histidine, 140 mM NaCl, pH 6.0) for further experiments.

### Biochemical Characterisation of GCase-BS

The enzymatic activity of the various GCase(-BS) constructs was determined using the fluorogenic substrate resorufin-β-D-glucopyranoside (res-β-glc: Sigma-Aldrich). GCase cleaves this substrate to glucose and resorufin. The product formation of resorufin was measured over time with excitation at a wavelength of 2=535 nm and emission at 2=595 nm in the assay buffer containing 50 mM citric acid pH 6.0, 50 mM KPi, 110 mM KCl, 10 mM NaCl, 1 mM MgCl2 and 1% DMSO at 37° C. Prior to the kinetic measurements, all assay components were pre-warmed to assay temperature and the fluorogenic substrate was kept in the dark. The reaction was started by adding GCase to a final concentration of 25 nM. Raw data from the fluorescence plate reader (Spectramax i3, Molecular Devices) were aggregated in Microsoft Excel and analysed using GraphPad Prism 8.4.2.

### FACS-Based Assessment of TfR Binding

The binding of the GCase-BS fusions was tested using mouse-TfR expressing cell line BA/F3 (DSMZ, ACC-300) or human-TfR expressing CHO cells (ATCC, CCL-61, transfected to stably overexpress human TfR). Briefly, suspension cells were harvested, counted, checked for viability and re-suspended at 2 million cells per ml in FACS buffer (PBS with 0.1% BSA). 100 μl of the cell suspension (containing 0.2 million cells) were incubated in round-bottom 96-well plates for 1 hour at 4° C. with increasing concentrations of the GCase fusions (10 pM to 1 μM). Cells were then washed twice with cold PBS/5% FBS, re-incubated for further 30 min at 4° C. in the dark with a labeled secondary antibody (PE-conjugated, goat-anti-hu IgG (Fc-spec.) from Jackson ImmunoResearch #109-116-170 at a dilution of 1:100, and washed twice with cold PBS/5% FBS. Fluorescence was analyzed by FACS using a BD FACSCanto™ II (Software FACS Diva and FlowJo 10.6.2). Binding curves and EC50 values were obtained using GraphPadPrism 7.

### Liquid Chromatography-Mass Spectrometry Analysis of GlcSph

Analytes and internal standards were purchased from Avanti Polar Lipids: D-glucosyl-β-1-1′-D-erythro-sphingosine (No. 860535) and D-glucosyl-β-1-1′-D-erythro-sphingosine-d5
as internal standard 1 (No. 860636): D-galactosyl-β-1-1′-D-erythro-sphingosine (No. 860537) and D-galactosyl-β-1-1′-D-erythro-sphingosine-d5 as internal standard 2 (No. 860637). For chromatography HPLC grade solvents as well as Millipore water was used. Acetonitrile (LiChrosoly No. 1.00030) and methanol (LiChrosoly No. 1.06007) were obtained from Supelco (Merck), ammonium acetate for mass spectrometry was purchased by Sigma-Aldrich (No. 73594).

Analyses were conducted on a LC-MS-MS system consisting of a Waters Xevo-TQ-S mass spectrometer connected to a complete Waters Acquity I-class UPLC system with a flow through needle sample manager using a mixture of acetonitrile/methanol/water 40/40/20 (v/v/v) as wash solvent. The autosampler temperature was set to 10° C.

Stock solutions for analytes and internal standards were prepared with a concentration of 1 mM in DMSO and kept at −20° C. For further spiking solutions acetonitrile/water 9/1 (v/v) was used as solvent. Calibration solutions were prepared by serial dilution in acetoni-trile/water 9/1 (v/v) containing 2% DMSO. The concentration range was from C1=10 μM to C9=0.0039 μM. Final calibration samples were made in a pooled tissue homogenate and prepared exactly the same way as brain and liver samples in order to avoid suppression effects deriving from the biological matrix.

Frozen tissues were weighed into 7 ml hard tissue homogenizing vials prefilled with ce-ramic beads (Bertin Cat. No. 03961-1-002.2 (CK28), supplied by LabForce AG, Switzerland or from Omni International, CatNo. 19-628) and homogenized with distilled water giving a final concentration of 100 mg tissue/ml. Samples, QC's and calibration samples were cleaned up by protein precipitation with methanol containing internal standards.

After centrifugation the supernatants were evaporated to dryness, reconstituted in ace-tonitrile/water 90/10 (v/v) with 1% DMSO and analyzed by LC-MS/MS. For calibration a linear regression function with 1/y weighting and excluding zero was used. The calibration range was from x+39 nM (C9) to x+1 μM (C1), where x is the endogenous substrate level in the pooled tissue homogenate. Absolute concentrations were calculated by dividing the peak area ratio analyte/internal standard by the slope of the calibration curve.

Samples were analyzed on a BEH glycan amide column (100×2.1 mm, 1.7 μm particle size, purchased from Waters, Switzerland) with a flow rate of 0.25 ml/min and an oven temperature of 30° C. Eluent A consisted of 100 mM ammonium acetate and for eluent B acetonitrile was used. Glycospecific separation was achieved by isocratic elution with 90% B followed by a washing step with 10% B and column reconditioning. The overall analysis time was 12 min.

The Xevo TQ-S instrument operated in positive ion electrospray mode with both quad-rupoles tuned to unit mass resolution using nitrogen as nebulization- and desolvation gas. The nebulizer gas flow was set to 150 l/h and the desolvation gas flow to 800 l/h with a temperature of 500° C. Argon was used as collision gas at a flow rate of 0.15 ml/min. Analytes and internal standards were detected by multiple reaction monitoring mode (MRM) following the transitions m/z 462.3 to 282.3 and m/z 467.3>287.3 at a cone voltage of 30 V and a collision energy of 18 V.

Cellular Assays to Assess Uptake. Lysosomal Activity and Potency of GCase-BS

GBA-deficient cell lines were used to determine uptake, lysosomal activity and potency of GCase-BS molecules.

To assess cellular uptake, the activity of GCase was determined from whole cell lysate. Cells were seeded at 5E4 cells/well into a 96-well plate and maintained at 37° C., 5% CO2, 85% humidity for 16-18 h. Cells were then treated with a range of concentrations of the various GCase-BS molecules for 2 h. Subsequently, cells were washed once with PBS and lysed in 30 μl lysis buffer (0.05 M citric acid, 0.05 M KH2PO4, 0.05 M K2HPO4, 0.11 M KCl, 0.01 M NaCl, 0.001 M MgCl2, pH 6.0 with 0.1% (v/v) TritonX-100, supplemented with freshly added protease inhibitor). 10 μl of cell lysate were mixed with 10 μl of 10 mM resorufin-β-glucopyranoside and baseline fluorescence was measured at to immediately. The build-up of fluorescent product (=resorufin) was measured after incubation for 2 h at 37° C. (hex=535 nm and % em=595 nm) indicating GCase activity. Data was normalised to WT cells.

To assess lysosomal activity, a fluorescence-quenched GCase substrate (FQ-7) was used that only emits light when GCase is hydrolysing it (and therefore releasing the quencher). Cells were seeded at 1E4 cells/well into a 96-well plate and maintained at 37° C., 5% CO2, 85% humidity for 16-18 h. Cells were then treated with a range of concentrations of the various GCase-BS molecules for 2 h. Subsequently, cells were washed with PBS and a mix of FQ-7 and SiR lyso kit were added for 1 h at 10 UM and 5 μM respectively. Cells were washed once more with PBS and Hoechst (2 μM final) was added for 3 min. Cells were imaged live using the Opera Phenix Plus (Perkin Elmer).

To assess potency of the GCase-BS molecules, cells were seeded at 2E4 cells/well into a 96-well plate and maintained at 37° C., 5% CO2, 85% humidity for 16-18 h. Cells were then treated with a range of concentrations of the various GCase-BS molecules for 48 h. Cells were washed once with PBS and lysed by adding distilled water and methanol containing internal analyte standards. Samples were evaporated to dryness, reconstituted in acetonitrile/water 90/10 (v/v) with 1% DMSO and analysed by LC-MS/MS. Lipids were simply quantified using the peak area ratio analyte/internal standard (=response).

To assess cellular localisation of the various constructs, immunocytochemistry labelling was performed in H4 cells. Cells were seeded at 5E3 cells/well into an imaging-compatible 96-well plate and treated with the molecules for 2 h. Subsequently, cells were washed once with PBS and fixed using 4% PFA for 10 min at RT. After fixation, cells were washed 3 times with PBS and blocked with 1% donkey serum, 1 mg/ml saponin, 0.75 mg/ml glycine in PBS for 2 h at RT. Primary antibody was incubated o/n at 4° C. in Ab dilution buffer (0.1% BSA, 1 mg/ml saponin in PBS). After 3 washes in PBS, secondary antibody was incubated in Ab dilution buffer for 2 h at RT. Cells were counterstained with DAPI to label the nuclei and imaged using a 40× objective at Opera Phenix Plus (Perkin Elmer).

### Assessment of Pharmacokinetics and -Dynamics of GCase-BS In Vivo

To determine plasma or brain tissue IgG concentrations in GBA+/+ mice treated with mGCase-mBS, samples were analysed with a generic ECLIA method specific for the human Ig/Fab CH1/kappa domain using a cobas e411 instrument under non-GLP conditions. Prior to analysis, brain tissue samples were mechanically homogenised in 500 μL of tissue extraction buffer containing protease inhibitors using the MagNA Lyser Homogenisator. In brief, samples, primary detection antibody (mAb anti-hFab (kappa)), secondary detection antibody (mAb anti-hFab (CH1)) and SA-beads were added stepwise to a detection vessel and incubated for 9 min in each step. Finally, the SA-beads-bound complex was detected by a measuring cell which numbers the counts of SA-beads in repeat. The counts were proportional to the analyte concentration in the test sample.

To determine the activity of mGCase-mBS in plasma, samples were analysed using an artificial substrate. Samples were mixed with 10 mM resorufin-β-glucopyranoside and baseline fluorescence was measured at to immediately. The build-up of fluorescent product (=resorufin) was measured after incubation for 2 h at 37° C. (λex=535 nm and λem=595 nm) indicating GCase activity. A defined standard curve of active mGCase in 10% matrix was used to determine amounts of active compound in plasma over time.

### NfL Analysis in Mouse Plasma

NFL detection in mouse plasma using Quanterix′ digital biomarker detection technology, Simoa®:

38 μl mouse plasma was mixed with 152 μl sample diluent from NF-light Kit (Quanterix #103186) and processed according to the manufacturers' instructions.

### Statistical Analysis

Statistical comparison of data was done using GraphPad Prism 6. Parametric tests (Student's two-tailed t-test for pairwise comparisons or ANOVA for multiple comparisons) were used. The p values and n values for all comparisons are indicated in each figure legend.

### Immunoprecipitation of Lysosomes (Lyso-IP Method) for Proteomic and Lipidomic Analysis

H4 cells (WT and GBA knock-out) stably expressing TMEM192-3XHA were seeded in 10/15 cm cell culture dish such that sufficient cells (≈20 million for proteomics and ≈50 million for lipidomics/replicate) are available on the day of lysosome isolation. GBA knock-out cells were treated with 1 nM hGCase-hBS for 24 h. On the day of lysosome isolation, cells were washed with ice-cold PBS, gently scraped, and centrifuged at 1000 g for 2 min. Cell pellets were resuspended in 1000 μl of ice-cold PBS and gently lysed using a rotary dounce ho-mogenizer at medium speed. Homogenate was centrifuged at 1000 g for 2 mins to remove cell debris. Part of the supernatant was preserved for quality control analysis. The remaining supernatant (≈900 μl) was incubated with 500 μl of anti-HA magnetic beads (Pierce/Thermo: 88836/88837) for 20 minutes at room temperature in a rotator shaker. Magnetic beads were separated using a magnetic rack and the flow-through was collected for quality control analysis. The magnetic beads carrying lysosomes were washed with ice-cold PBS. For the proteomic samples, 200 μl of 1× RIPA buffer was added to magnetic beads carrying lysosomes and heated for 5 minutes at 95° C. The resulting protein samples from lysosomes were acetone precipitated and used for further analysis. For the lipidomic samples, lysosomes were separated from magnetic beads using competitive elution due to the presence of high concentration of HA peptide. Magnetic beads carrying lysosomes were incubated with 500 μl of 1 mg/ml HA peptide (in PBS) and incubated for 15 min at 37° C. Magnetic beads were removed using a magnetic rack and the remaining lysosome containing samples were immediately frozen at −80° C. for further analysis. For corresponding whole-cell lysate samples, cells were seeded in 10 cm culture dish (≈8 million cells per replicate) and treated with hGCase-hBS wherever applicable. Cells were gently scraped and cell pellets were collected by centrifugation at 1000 g for 2 min.

### Proteomics Sample Preparation

Samples were denatured using Biognosys' Denature Buffer, reduced using Biognosys' Reduction Solution for 60 min at 37° C. and alkylated using Biognosys' Alkylation Solution for 30 min at room temperature in the dark. Subsequently, digestion to peptides was carried out using 0.5 μg of trypsin (Promega) per sample overnight at 37° C. . . . Peptides were desalted using a C18 MicroSpin plate (The Nest Group) according to the manufacturer's instructions and dried down using a SpeedVac system Peptides were resuspended in 20 μl LC solvent A (1% acetonitrile, 0.1% formic acid (FA) and spiked with Biognosys' iRT kit calibration peptides. Peptide concentrations were determined using a UV/VIS Spectrometer (SPECTROstar Nano, BMG Labtech).

### HRM Mass Spectrometry Acquisition for Proteomics

For DIA LC-MS/MS measurements, 1 μg of peptides per sample were injected to an in house packed reversed phase column (PicoFrit emitter with 75 μm inner diameter, 60 cm length and 10 μm tip from New Objective, packed with 1.7 μm Charged Surface Hybrid C18 particles from Waters) on a Thermo Scientific™ EASY-nLC™ 1200 nano liquid chromatography system connected to a Thermo Scientific™ Q Exactive™ HF mass spectrometer equipped with a Nanospray Flex™ Ion Source. LC solvents were A: 1% acetonitrile in water with 0.1% FA; B: 20% water in acetonitrile with 0.1% FA. The nonlinear LC gradient was 1-59% solvent B in 55 minutes followed by 59-90% B in 10 seconds, 90% B for 8 minutes, 90%-1% B in 10 seconds and 1% B for 5 minutes at 60° C. and a flow rate of 250 nl/min The DIA method consisted of one full range MS1 scan and 21 DIA segments was adopted from Bruderer et al., 2017.

### Proteomics Data Analysis

Proteins with low intensities and NA values were filtered, and subsequently analyzed using the edgeR Bioconductor package. Protein name to HUGO gene symbol mapping was performed using Bioconductors org.Hs.eg.db package. Libraries were normalized using TMM to remove composition bias, and we fitted a negative binomial generalized log-linear model to the log 2 intensities of each protein, taking into account genewise, trended and common dis-persion estimates. Testing for differential expression of proteins between comparison groups was tested with a log likelihood ratio test. For comparison and ranking of interesting hits resulting from the contrasts of interest, we introduced a comparison metric which combines sig-nificance and change in protein abundance: metric=−Log 10 (p.value)*Log 2(FC). A metric threshold of 6 was used for filtering the top differentially expressed proteins.

### Lipid Extraction for Mass Spectrometry Lipidomics

Mass spectrometry-based lipid analysis was performed by Lipotype GmbH (Dresden, Germany) as described (Sampaio et al. 2011). Lipids were extracted using a two-step chloro-form/methanol procedure (Ejsing et al. 2009). Samples were spiked with internal lipid standard mixture containing: cardiolipin 14:0/14:0/14:0/14:0 (CL), ceramide 18:1:2/17:0 (Cer), di-acylglycerol 17:0/17:0 (DAG), hexosylceramide 18:1:2/12:0 (HexCer), lyso-phosphatidate 17:0 (LPA), lyso-phosphatidylcholine 12:0 (LPC), lyso-phosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0 (PG), phosphatidylinositol 16:0/16:0 (PI), phosphatidylserine 17:0/17:0 (PS), cholesterol ester 20:0 (CE), sphingomyelin 18:1:2/12:0:0 (SM), triacylglycerol 17:0/17:0/17:0 (TAG) and cholesterol D6 (Chol). After extraction, the organic phase was transferred to an infusion plate and dried in a speed vacuum concentrator. 1st step dry extract was re-suspended in 7.5 mM ammonium acetate in chloroform/methanol/propanol (1:2:4, V:V:V) and 2nd step dry extract in 33% ethanol solution of methylamine in chloroform/methanol (0.003:5:1; V:V:V). All liquid handling steps were performed using Hamilton Robotics STARlet robotic platform with the Anti Drop-let Control feature for organic solvents pipetting.

### MS Data Acquisition for Lipidomics

Samples were analyzed by direct infusion on a QExactive mass spectrometer (Thermo Scientific) equipped with a TriVersa NanoMate ion source (Advion Biosciences). Samples were analyzed in both positive and negative ion modes with a resolution of Rm/z=200=280000 for MS and Rm/z=200=17500 for MSMS experiments, in a single acquisition. MSMS was triggered by an inclusion list encompassing corresponding MS mass ranges scanned in 1 Da increments (Surma et al. 2015). Both MS and MSMS data were combined to monitor CE, DAG and TAG ions as ammonium adducts: PC, PC O—, as acetate adducts; and CL, PA, PE, PE O—, PG, PI and PS as deprotonated anions. MS only was used to monitor LPA, LPE, LPE O—, LPI and LPS as deprotonated anions; Cer, HexCer, SM, LPC and LPC O— as acetate adducts and cholesterol as ammonium adduct of an acetylated derivative (Liebisch et al. 2006).

### Lipidomics Data Analysis

Data were analysed with in-house developed lipid identification software based on LipidXplorer (Herzog et al. 2012, 2011). Data post-processing and normalisation were performed using an in-house developed data management system. Only lipid identifications with a signal-to-noise ratio>5, and a signal intensity 5-fold higher than in corresponding blank samples were considered for further data analysis. Data were analysed with R version 4.0.3 (2020-10-10) (RCore Team 2017) using tidyverse packages (version 1.3.0) (Wickham 2019) and bioconductor pcaMethods (Stacklies et al. 2007). Lipids were quantified in molar frac-tions (molp) and standardized to the total lipid amount per sample due to large differences in the total lipid amounts across samples. A 70% occupational threshold was applied, yielding 1196 lipids to be compared. Differential lipidomics analysis was performed using an unpaired t-test between the test groups (GBA-KO vs WT and KOE vs KO in both lysosomal and whole cell lysates). Fold changes between comparison groups are defined as the Log 2 fold change of the means. We used the same combined p-value/log FC metric as for the lipid contrasts to compare the top hits. For displaying the top hits between GBA-KO vs WT and KOE vs KO comparison (FIG. 4D, Supp. FIG. B), we applied a metric threshold of 1 for both directions.

### REFERENCES

1. Braulke, T. & Bonifacino, J. S. Sorting of lysosomal proteins. Biochimica Et Bio-physica Acta Bba-Mol Cell Res 1793, 605-614 (2009).
2. Boustany, R. M. N. Lysosomal storage diseases—the horizon expands. Nat Rev Neurol 9, 583-598 (2013).
3. Boer, D. E. C., Smeden, J. van, Bouwstra, J. A. & Aerts, J. M. F. G. Glucocerebrosidase: Functions in and Beyond the Lysosome. J Clin Medicine 9, 736 (2020).
4. Aflaki, E., Westbroek, W. & Sidransky, E. The Complicated Relationship between Gaucher Disease and Parkinsonism: Insights from a Rare Disease. Neuron 93, 737-746 (2017).
5. Beutler, E., and Grabowski, GA. (2001). Gaucher disease. In The Metabolic and Molecular Basis of Inherited Disease, S. CR, B. A L, S. WS, and V. D, eds. (New York: McGraw-Hill), pp. 3635-3668.
6. Lal, T. R. et al. The natural history of type 2 Gaucher disease in the 21st century: A retrospective study. Neurology 95, e2119-e2130 (2020).
7. Weiss, K. et al. The clinical management of type 2 Gaucher disease. Mol Genet Metab 114, 110-122 (2015).
8. Lal. T. R. & Sidransky, E. The Spectrum of Neurological Manifestations Associated with Gaucher Disease. Dis 5, 10 (2017).
9. Sidransky, E. & Lopez, G. The link between the GBA gene and parkinsonism. Lancet Neurology 11, 986-998 (2012).
10. Liu, G. et al. Specifically neuropathic Gaucher's mutations accelerate cognitive decline in Parkinson's. Ann Neurol 80, 674-685 (2016).
11. Migdalska-Richards, A. & Schapira, A. H. V. The relationship between glucocerebrosidase mutations and Parkinson disease. J Neurochem 139, 77-90 (2016).
12. Huebecker, M. et al. Reduced sphingolipid hydrolase activities, substrate accumulation and ganglioside decline in Parkinson's disease. Mol Neurodegener 14, 40 (2019).
13. Orvisky, E. et al. Glucosylsphingosine Accumulation in Mice and Patients with Type 2 Gaucher Disease Begins Early in Gestation. Pediatr Res 48, 233-237 (2000).
14. Aerts, J. M. F. G. et al. Glycosphingolipids and lysosomal storage disorders as illus-trated by gaucher disease. Curr Opin Chem Biol 53, 204-215 (2019).
15. Fishman, J. B., Rubin, J. B., Handrahan, J. V., Connor, J. R. & Fine, R. E. Receptor-mediated transcytosis of transferrin across the blood-brain barrier. J Neurosci Res 18, 299-304 (1987).
16. Descamps, L., Dehouck, M. P., Torpier, G. & Cecchelli, R. Receptor-mediated transcytosis of transferrin through blood-brain barrier endothelial cells. Am J Physiol-heart C 270, H1149-H1158 (1996).
17. Bien-Ly, N. et al. Transferrin receptor (TfR) trafficking determines brain uptake of TfR antibody affinity variants. J Exp Medicine 211, 233-244 (2014).
18. Niewoehner, J. et al. Increased Brain Penetration and Potency of a Therapeutic Antibody Using a Monovalent Molecular Shuttle. Neuron 81, 49-60 (2014).
19. Ridgway, J. B. B., Presta, L. G. & Carter, P. ‘Knobs-into-holes’ engineering of antibody CH3 domains for heavy chain heterodimerization. Protein Eng Des Sel 9, 617-621 (1996).
20. Westbroek, W. et al. A new glucocerebrosidase-deficient neuronal cell model provides a tool to probe pathophysiology and therapeutics for Gaucher disease. Dis Model Mech 9, 769-778 (2016).
21. Gündner, A. L. et al. Generation of a homozygous GBA deletion human embryonic stem cell line. Stem Cell Res 23, 122-126 (2017).
22. Polinski, N. K. et al. Decreased glucocerebrosidase activity and substrate accumulation of glycosphingolipids in a novel GBA1 D409V knock-in mouse model. Plos One 16, e0252325 (2021).
23. Pohl, S., Marschner, K., Storch, S. & Braulke, T. Glycosylation- and phosphoryla-tion-dependent intracellular transport of lysosomal hydrolases. Biol Chem 390, 521-527 (2009).
24. Fedele, S. et al. Expansion of human midbrain floor plate progenitors from induced pluripotent stem cells increases dopaminergic neuron differentiation potential. Sci Rep-uk 7, 6036 (2017).
25. Crook, Z. R. et al. A TfR-Binding Cystine-Dense Peptide Promotes Blood-Brain Barrier Penetration of Bioactive Molecules. J Mol Biol 432, 3989-4009 (2020).
26. Yu, Y. J. et al. Boosting Brain Uptake of a Therapeutic Antibody by Reducing Its Affinity for a Transcytosis Target. Sci Transl Med 3, 84ra44-84ra44 (2011).
27. Abu-Remaileh, M. et al. Lysosomal metabolomics reveals V-ATPase- and mTOR-dependent regulation of amino acid efflux from lysosomes. Science 358, 807-813 (2017).
28. Thelen, A. M. & Zoncu, R. Emerging Roles for the Lysosome in Lipid Metabolism. Trends Cell Biol 27, 833-850 (2017).
29. Singh, J. et al. Systematic Comparison of Strategies for the Enrichment of Lysosomes by Data Independent Acquisition. J Proteome Res 19, 371-381 (2020).
30. Horvath, I. et al. Co-aggregation of pro-inflammatory S100A9 with α-synuclein in Parkinson's disease: ex vivo and in vitro studies. J Neuroinflamm 15, 172 (2018).
31. Toleikis, Z. et al. S100A9 Alters the Pathway of Alpha-Synuclein Amyloid Aggregation. Int J Mol Sci 22, 7972 (2021).
32. Ren, Y., Jiang, H., Ma, D., Nakaso, K. & Feng, J. Parkin degrades estrogen-related receptors to limit the expression of monoamine oxidases. Hum Mol Genet 20, 1074-1083 (2011).
33. Schiffer, V. et al. Characterization of the visceral and neuronal phenotype of 4L/PS-NA mice modeling Gaucher disease. Plos One 15, e0227077 (2020).
34. Sun, Y., Quinn, B., Witte, D. P. & Grabowski, G. A. Gaucher disease mouse models: point mutations at the acid β-glucosidase locus combined with low-level prosaposin expression lead to disease variants. J Lipid Res 46, 2102-2113 (2005).
35. Udayar, V., Chen, Y., Sidransky, E. & Jagasia, R. Lysosomal dysfunction in neurodegeneration: emerging concepts and methods. Trends Neurosci (2022) doi: 10.1016/j.tins.2021.12.004.
36. Futerman, A. H. & Meer, G. van. The cell biology of lysosomal storage disorders. Nat Rev Mol Cell Bio 5, 554-565 (2004).
37. Hoffmann, B. & Mayatepek, E. Neurological Manifestations in Lysosomal Storage Disorders—From Pathology to First Therapeutic Possibilities. Neuropediatrics 36, 285-289 (2005)
38. Sardi, S. P. et al. Augmenting CNS glucocerebrosidase activity as a therapeutic strategy for parkinsonism and other Gaucher-related synucleinopathies. Proc National Acad Sci 110, 3537-3542 (2013).
39. Ullman, J. C. et al. Brain delivery and activity of a lysosomal enzyme using a blood-brain barrier transport vehicle in mice. Sci Transl Med 12, eaay 1163 (2020).
40. Kariolis, M. S. et al. Brain delivery of therapeutic proteins using an Fc fragment blood-brain barrier transport vehicle in mice and monkeys. Sci Transl Med 12, eaay 1359 (2020).
41. Mullard, A. Blood-brain barrier-traversing biologic secures regulatory approval, in Japan. Nat Rev Drug Discov 20, 332-332 (2021).
42. Sonoda, H. et al. A Blood-Brain-Barrier-Penetrating Anti-human Transferrin Receptor Antibody Fusion Protein for Neuronopathic Mucopolysaccharidosis II. Mol Ther 26, 1366-1374 (2018).
43. Logan, T. et al. Rescue of a lysosomal storage disorder caused by Grn loss of function with a brain penetrant progranulin biologic. Cell 184, 4651-4668.e25 (2021).
44. Liou, B., Haffey, W. D., Greis, K. D. & Grabowski, G. A. The LIMP-2/SCARB2 Binding Motif on Acid β-Glucosidase. J Biol Chem 289, 30063-30074 (2014).
45. Braulke, T. Type-2 IGF Receptor: A Multi-Ligand Binding Protein. Horm Metab Res 31, 242-246 (1999).
46. Ghosh, P., Dahms, N. M. & Komfeld, S. Mannose 6-phosphate receptors: new twists in the tale. Nat Rev Mol Cell Bio 4, 202-213 (2003).
47. Benz, J., Ebeling, M., Jagasia, R., Pfahler, N., and Thoma, R. (2021). Glucocerebrosidase mutants. WO2021048034A1.
48. Do, H. (2013). Variant, recombinant beta-glucocerebrosidase protein with increased stability and increased retained catalytic activity. US2013344054A1.
49. Han, T. U., Sam, R. & Sidransky, E. Small Molecule Chaperones for the Treatment of Gaucher Disease and GBA1-Associated Parkinson Disease. Frontiers Cell Dev Biology 8, 271 (2020).
50. Narita, A. et al. Ambroxol chaperone therapy for neuronopathic Gaucher disease: A pilot study. Ann Clin Transl Neur 3, 200-215 (2016).
51. Maegawa, G. H. B. et al. Identification and Characterization of Ambroxol as an Enzyme Enhancement Agent for Gaucher Disease*. J Biol Chem 284, 23502-23516 (2009).
52. Ishay, Y. et al. Combined beta-glucosylceramide and ambroxol hydrochloride in patients with Gaucher related Parkinson disease: From clinical observations to drug develop-ment. Blood Cells Mol Dis 68, 117-120 (2018).
53. Byrne, B., Donohoe, G. G. & O'Kennedy, R. Sialic acids: carbohydrate moieties that influence the biological and physical properties of biopharmaceutical proteins and living cells. Drug Discov Today 12, 319-326 (2007).
54. Lindhout, T. et al. Site-specific enzymatic polysialylation of therapeutic proteins using bacterial enzymes. Proc National Acad Sci 108, 7397-7402 (2011).
55. Terekhov, S. S. et al. Chemical Polysialylation and In Vivo Tetramerization Improve Pharmacokinetic Characteristics of Recombinant Human Butyrylcholinesterase-Based Bioscavengers. Acta Naturae 7, 136-41 (2015).
56. Farfel-Becker, T., Do, J., Tayebi, N. & Sidransky, E. Can GBA1-Associated Parkinson Disease Be Modeled in the Mouse? Trends Neurosci 42, 631-643 (2019).
57. Loeffler, T., Schilcher, I., Flunkert, S. & Hutter-Paier, B. Neurofilament-Light Chain as Biomarker of Neurodegenerative and Rare Diseases With High Translational Value. Front Neurosci-switz 14, 579 (2020).
58. Tayebi, N., Lopez, G., Do, J. & Sidransky, E. Pro-cathepsin D, Prosaposin, and Progranulin: Lysosomal Networks in Parkinsonism. Trends Mol Med 26, 913-923 (2020).
59. Peterschmitt, M. J. et al. Pharmacokinetics, Pharmacodynamics, Safety, and Tolera-bility of Oral Venglustat in Healthy Volunteers. Clin Pharm Drug Dev 10, 86-98 (2021).
60. Sardi. S. P., Cedarbaum. J. M. & Brundin. P. Targeted Therapies for Parkinson's Disease: From Genetics to the Clinic. Movement Disord 33, 684-696 (2018).

What is claimed is:

1. A fusion protein comprising:
a) a lysosomal protein,
b) a Fc region of an antibody and
c) an antibody fragment targeting the transferrin receptor, wherein the antibody fragment has a monovalent binding mode.

2. The fusion protein of claim 1, wherein the lysosomal protein is a β-Glucocerebrosidase (Gcase) protein, preferably the human Gcase protein or a variant thereof.

3. The fusion protein of claim 1 or 2, wherein the Fc region of an antibody is the Fc region of an IgG antibody, preferably an IgG1 antibody.

4. The fusion protein of claims 1-3, wherein the Fc region is devoid of Fc receptor gamma binding.

5. The fusion protein of claims 1-4, wherein the antibody fragment targeting the transferrin receptor is selected from the group consisting of Fv, Fab, Fab′, Fab′-SH, F(ab′) diabodies, linear antibodies, single-chain antibody molecule such as e.g. scFv, scFab, cross Fab and single domain antibodies (dAbs).

6. The fusion protein of claims 1-5, wherein one chain of the Fc region is fused at its N-terminal end to the C-terminal end of the lysosomal protein and the second Fc chain is fused at its C-terminal end to the antibody fragment targeting the transferrin receptor.

7. The fusion protein of claims 1-6, wherein the two Fc chains form a dimer using the knob-into-hole technology.

8. The fusion protein of claims 1-7, wherein the fusion protein comprises two protein chains:
a. a first protein chain comprising the lysosomal protein fused at its C-terminal end to a first chain of the Fc region comprising the knob-into-hole technology,
b. the second protein chain comprising the second chain of the Fc region comprising the knob-into-hole technology fused at its C-terminal end to the scFab antibody fragment targeting the transferrin receptor.

9. The fusion protein of claims 1-8, wherein the human Gcase protein has the amino acid sequence set forth in Seq. Id. No. 1.

10. The fusion protein of claim 8, wherein the first protein chain has the amino acid sequence set forth in Seq. Id. No. 2 and the second single chain protein has the amino acid sequence set forth in Seq. Id. No. 3.

11. An isolated nucleic acid molecule encoding the fusion protein of claims 1-11.

12. The isolated nucleic acid of claim 11, wherein the nucleic acid is a circular RNA.

13. A host cell comprising the isolated nucleic acid molecule of claim 11 or 12.

14. A pharmaceutical formulation comprising the fusion protein of claims 1-10.

15. The fusion protein of claims 1-10 for use as a medicament.

16. The fusion protein of claims 1-10 for use in the treatment of a neurodegenerative disorder.


SEQUENCE LISTING (16 sequences)

SEQ ID NO: 1 - protein, 497 aa, Homo sapiens [sequence omitted]

SEQ ID NO: 2 - protein, 735 aa, Homo sapiens
  REGION 1..497: note = MISC_FEATURE - Human GCase R534H
  REGION 498..508: note = MISC_FEATURE - Peptide linker
  REGION 509..735: note = MISC_FEATURE - human IgG1 Fc chain: KnobC1, LALA, P329G
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWHRQGSGGGGSGGSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 721 aa, Homo sapiens
  REGION 1..226: note = MISC_FEATURE - IgG1 Fc LALA, P329G, HYRF, HoleC1
  REGION 227..244: note = MISC_FEATURE - Peptide linker
  REGION 245..474: note = MISC_FEATURE - BS1026 scFab chain
  REGION 475..506: note = MISC_FEATURE - Peptide linker
  REGION 507..721: note = MISC_FEATURE - BS1026 scFab chain
ADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGGGSGGGGSGGGGSGGGGSQSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGAIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 4 - protein, 710 aa, Homo sapiens
  REGION 1..225: note = MISC_FEATURE - Fc human IgG1
  REGION 226..245: note = MISC_FEATURE - Peptide linker
  REGION 246..460: note = MISC_FEATURE - DP47 scFab chain
  REGION 461..492: note = MISC_FEATURE - Peptide linker
  REGION 493..710: note = MISC_FEATURE - DP47 scFab chain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 5 - protein, 515 aa, Mus musculus
AQPCIPKSFGYSSVVCVCNASYCDSLDPVTLPALGTFSRYESTRRGRRMELSVGAIQANRTGTGLLLTLQPEKKFQKVKGFGGAMTDATALNILALSPPTQKLLLRSYFSTNGIEYNIIRVPMASCDFSIRVYTYADTPNDFQLSNFSLPEEDTKLKIPLIHQALKMSSRPISLFASPWTSPTWLKTNGRVNGKGSLKGQPGDIFHQTWANYFVKFLDAYAKYGLRFWAVTAENEPTAGLFTGYPFQCLGFTPEHQRDFISRDLGPALANSSHDVKLLMLDDQRLLLPRWAEVVLSDPEAAKYVHGIAVHWYMDFLAPAKATLGETHRLFPNTMLFASEACVGSKFWEQSVRLGSWDRGMQYSHSIITNLLYHVTGWTDWNLALNPEGGPNWVRNFVDSPIIVDIPKDAFYKQPMFYHLGHFSKFIPEGSQRVALVASESTDLETVALLRPDGSAVVVVLNRSSEDVPLTISDPDLGFLETVSPGYSIHTYLWRRQGSLPETGGSGGHHHHHHHH

SEQ ID NO: 6 - protein, 732 aa, Mus musculus
AQPCIPKSFGYSSVVCVCNASYCDSLDPVTLPALGTFSRYESTRRGRRMELSVGAIQANRTGTGLLLTLQPEKKFQKVKGFGGAMTDATALNILALSPPTQKLLLRSYFSTNGIEYNIIRVPMASCDFSIRVYTYADTPNDFQLSNFSLPEEDTKLKIPLIHQALKMSSRPISLFASPWTSPTWLKTNGRVNGKGSLKGQPGDIFHQTWANYFVKFLDAYAKYGLRFWAVTAENEPTAGLFTGYPFQCLGFTPEHQRDFISRDLGPALANSSHDVKLLMLDDQRLLLPRWAEVVLSDPEAAKYVHGIAVHWYMDFLAPAKATLGETHRLFPNTMLFASEACVGSKFWEQSVRLGSWDRGMQYSHSIITNLLYHVTGWTDWNLALNPEGGPNWVRNFVDSPIIVDIPKDAFYKQPMFYHLGHFSKFIPEGSQRVALVASESTDLETVALLRPDGSAVVVVLNRSSEDVPLTISDPDLGFLETVSPGYSIHTYLWRRQGSLPETGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 7 - protein, 711 aa, Mus musculus
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 8 - DNA, 20 bp, synthetic construct
  modified_base 2: mod_base = OTHER; note = uracil
  source 1..20: mol_type = other DNA
  modified_base 5: mod_base = OTHER; mod_base = OTHER; note = Uracil
  modified_base 8: mod_base = OTHER; note = Uracil
  modified_base 10..14: mod_base = OTHER; note = Uracil
  modified_base 16..17: mod_base = OTHER; note = Uracil
  modified_base 20: mod_base = OTHER; note = Uracil
gtgatcgtctttttcttgat

SEQ ID NO: 9 - DNA, 20 bp, synthetic construct
  source 1..20: mol_type = other DNA
  modified_base 4: mod_base = OTHER; note = Uracil
  modified_base 7: mod_base = OTHER; note = Uracil
  modified_base 13: mod_base = OTHER; note = Uracil
aaatgctgacaataacacaa

SEQ ID NO: 10 - DNA, 20 bp, synthetic construct
  modified_base 4: mod_base = OTHER; note = Uracil
  source 1..20: mol_type = other DNA
  modified_base 10: mod_base = OTHER; note = Uracil
  modified_base 16: mod_base = OTHER; note = Uracil
  modified_base 19: mod_base = OTHER; note = Uracil
agatggcgataacagtcatg

SEQ ID NO: 11 - DNA, 20 bp, synthetic construct
  modified_base 3: mod_base = OTHER; note = Uracil
  source 1..20: mol_type = other DNA
  modified_base 13: mod_base = OTHER; note = Uracil
  modified_base 16: mod_base = OTHER; note = Uracil
aatcaacaaaagtaatggga

SEQ ID NO: 12 - DNA, 20 bp, synthetic construct
  modified_base 1..2: mod_base = OTHER; note = Uracil
  source 1..20: mol_type = other DNA
  modified_base 8: mod_base = OTHER; note = Uracil
  modified_base 10: mod_base = OTHER; note = Uracil
ttcagggtgtgccgggaagc

SEQ ID NO: 13 - DNA, 20 bp, synthetic construct
  source 1..20: mol_type = other DNA
  modified_base 1: mod_base = OTHER; note = Uracil
  modified_base 7..9: mod_base = OTHER; note = Uracil
  modified_base 18: mod_base = OTHER; note = Uracil
  modified_base 20: mod_base = OTHER; note = Uracil
tacagctttgagagcactgt

SEQ ID NO: 14 - DNA, 20 bp, synthetic construct
  source 1..20: mol_type = other DNA
  modified_base 1..2: mod_base = OTHER; note = Uracil
  modified_base 6..7: mod_base = OTHER; note = Uracil
  modified_base 9: mod_base = OTHER; note = Uracil
ttgaattgtgcaggtaacga

SEQ ID NO: 15 - DNA, 20 bp, synthetic construct
  source 1..20: mol_type = other DNA
  modified_base 3: mod_base = OTHER; note = Uracil
  modified_base 5: mod_base = OTHER; note = Uracil
  modified_base 10: mod_base = OTHER; note = Uracil
  modified_base 12: mod_base = OTHER; note = Uracil
  modified_base 20: mod_base = OTHER; note = Uracil
cgtgtcccatgtgaagaagt

SEQ ID NO: 16 - DNA, 20 bp, synthetic construct
  source 1..20: mol_type = other DNA
  modified_base 3: mod_base = OTHER; note = Uracil
  modified_base 7: mod_base = OTHER; note = Uracil
cgtcggtggcaccgcagagg